(19) United States
(12) Patent Application Publication
(10) Pub. No.: US 2022/0275409 A1
(43) Pub. Date: Sep. 1, 2022

(54) A GENETICALLY MODIFIED FUNGUS AND METHODS AND USES RELATED THERETO

(71) Applicant: FF-FUTURE OY, Salo (FI)

(72) Inventors: Simo ELLILÄ, VTT (FI); Marja ILMÉN, VTT (FI); Maija-Leena VEHKOMÄKI, VTT (FI)

(21) Appl. No.: 17/638,080

(22) PCT Filed: Aug. 28, 2020
(86) PCT No.: PCT/FI2020/050557

§ 371 Date: Feb. 24, 2022

(30) Foreign Application Priority Data
Aug. 30, 2019 (FI) 20195716

(51) Int. Cl. C12P 7/56; C12N 15/80; C12N 15/81; C12N 1/16; C12N 9/04; C12N 9/40

(52) U.S. Cl. CPC C12P 7/56; C12N 15/80; C12N 15/81; C12N 1/16; C12N 9/0006; C12N 9/2465; C12Y 101/01027; C12Y 302/01022

(57) ABSTRACT

The present invention relates to the fields of industrial biotechnology, renewable raw materials and microbial production organisms. Specifically, the invention relates to a method of producing lactic acid or lactate or one or more products selected from the group consisting of polymers, polyesters and polylactic acids. Still, the present invention relates to a genetically modified fungus comprising increased specific enzyme activities, a method of preparing said genetically modified fungus, and use of said fungus for producing lactic acid, lactate or polymers.

### FIELD OF THE INVENTION

The present invention relates to the fields of industrial biotechnology, renewable raw materials and microbial production organisms. Specifically, the invention relates to a method of producing lactic acid or lactate or one or more products selected from the group consisting of polymers, polyesters and polylactic acids. Still, the present invention relates to a genetically modified fungus comprising increased specific enzyme activities, a method of preparing said genetically modified fungus, and use of said fungus for producing lactic acid, lactate or polymers.

### BACKGROUND OF THE INVENTION

Lactic acid fermentation is an anaerobic metabolic process by which e.g. glucose and other hexoses (six-carbon sugars) or disaccharides of six-carbon sugars (e.g. sucrose or lactose) are converted into energy and lactic acid. Lactic acid is currently produced from corn starch in the USA and other sources of sugar such as sugar beet and sugarcane elsewhere. Said starch and sugar sources mainly comprise simple carbohydrates. Lactic acid is produced for food use, but also as a precursor for poly lactic acid (PLA) production. PLA is a renewable polymer that is increasingly used in the manufacture of bioplastics. For PLA production optically pure isomers are required which are generally not produced by wild type microbes.

Cheaper and ecologically compatible feedstocks for lactic acid production are needed. As an example, bacteria Lactobacillus salivarius have been utilized for conversion of soy molasses into lactic acid (Montelongo J et al., 1993, Journal of food science, vol. 58, 863-866). However, there remains a significant unmet need for effective fungus capable of converting complex carbohydrates such as galacto-oligosaccharides into lactic acid.

### BRIEF DESCRIPTION OF THE INVENTION

The objects of the invention, namely obtaining effective methods for producing lactic acid and/or lactate as well as obtaining a fungus capable of effectively converting carbohydrates into lactic acid and/or lactate, are achieved by utilizing genetic modifications of a fungus.

The present invention enables overcoming the defects of the prior art including but not limited to lack of a fungus capable of converting complex carbohydrates (including but not limited to carbohydrates of soy molasses) into lactic acid. Indeed, the fungus and method of the present invention allow use of alternative carbon substrates compared to e.g. corn starch and sucrose, for lactic acid production in industrial scale. Thus, the present invention provides value to ecological development by allowing utilization of industrial side streams comprising complex carbohydrates.

Currently the cost of e.g. PLA is not competitive with synthetic plastics. However, the present invention allows reduction of production costs of polymers such as PLA or polyesters.

Surprisingly the fungus and methods of the present invention enable production of pure L-lactic acid isomer with high yield, titer and productivity for industrially economical operation.

The present invention relates to a method of producing lactic acid and/or lactate, said method comprising
providing a fungus that has been genetically modified to increase lactate dehydrogenase enzyme and alfa-galactosidase enzyme activities,
culturing said fungus in a medium comprising a carbon substrate (e.g. a carbon substrate comprising galacto-oligosaccharides) to obtain lactic acid and/or lactate.

Also, the present invention relates to a genetically modified fungus comprising increased lactate dehydrogenase enzyme and alfa-galactosidase enzyme activities.

Still, the present invention relates to a method of preparing the genetically modified fungus of the present invention comprising increased lactate dehydrogenase enzyme and alfa-galactosidase enzyme activities, wherein said method comprises providing a fungus and genetically modifying the fungus to increase lactate dehydrogenase enzyme and alfa-galactosidase enzyme activities.

Still furthermore, the present invention relates to use of the fungus of the present invention comprising increased lactate dehydrogenase enzyme and alfa-galactosidase enzyme activities, for producing lactic acid and/or lactate or for producing polymers, optionally polyesters or polylactic acids.

And still furthermore, the present invention relates to a method of producing one or more products selected from the group consisting of polymers, polyesters and polylactic acids, said method comprising culturing the genetically modified fungus of the present invention (comprising increased lactate dehydrogenase enzyme and alfa-galactosidase enzyme activities) in a carbon substrate, e.g. galacto-oligosaccharides, containing medium to produce lactic acid, recovering the resulting lactic acid and utilizing the recovered lactic acid in production of polymers, polyesters and/or polylactic acids.

Other objects, details and advantages of the present invention will become apparent from the following drawings, detailed description and examples.

### BRIEF DESCRIPTION OF THE DRAWINGS

FIG. 1 shows the growth of various wild-type fungal strains of Kluyveromyces marxianus and Candida apicola using galactose as sole carbon source. The growth of strains was assessed by quantifying OD₆₀₀.

FIG. 2 shows the growth of four fungal strains expressing lactate dehydrogenase (ldh) using galactose as sole carbon source. The growth of strains was assessed by quantifying OD₆₀₀.

FIG. 3 shows the growth of S. cerevisiae strains expressing different genes coding for α-galactosidase on a SC-Ura medium with 1% melibiose or raffinose as carbon source. The strains were cultivated overnight in a 4 ml culture volume in 24-well plates, with 220 rpm shaking, at 30° C.

FIG. 4 shows ethanol titers (g/L) quantified by HPLC from 24 h cultures on 1:3 diluted soy molasses of parental strain (VTT-C-02453 ura3Δ/ura3Δ) and derived strains expressing different α-galactosidases.

FIG. 5 shows residual sugars (g/L) quantified by HPLC from 24 h cultures on 1:3 diluted soy molasses of parental strain (VTT-C-02453 ura3Δ/ura3Δ) and derived strains expressing different α-galactosidases.

FIG. 6 shows lactic acid (g/L) quantified by HPLC from bioreactor cultures of S. cerevisiae E79-4 and derived strains expressing different α-galactosidases. The strains were grown using soy molasses as sole carbon source.

FIG. 7 shows residual galacto-oligosaccharides (g/L) quantified from bioreactor cultures of S. cerevisiae E79-4 and derived strains expressing different α-galactosidases. The strains were grown using soy molasses as sole carbon source. The results are reported as the sum of the concentrations of raffinose, stachyose, verbascose, melibiose, manninotriose and manninotetraose.

FIG. 8 shows maps of the plasmids used in examples 1-4.

FIG. 9 reveals residual tetra- and tri-saccharides quantified from shake flask cultures using soy molasses as carbon source of modified yeast strain VTT C-191026 and strains expressing additional copies of different α-galactosidase genes.

FIG. 10 reveals produced lactic acid and residual tri- and di-saccharides quantified from shake flask cultures using soy molasses as carbon source of modified yeast strain VTT C-191026 and a modified P. kudriavzevii strain VTT C-201040.

FIG. 11 shows maps of the plasmids used in example 6.

### SEQUENCE LISTING

SEQ ID NO:1: an amino acid sequence of an alfa-galactosidase (A. niger aglC)
SEQ ID NO:2: an amino acid sequence of an alfa-galactosidase (T. reesei agl1)
SEQ ID NO:3: an amino acid sequence of an alfa-galactosidase (Rhizomucor miehei GAL36)
SEQ ID NO:4: an amino acid sequence of an alfa-galactosidase (Gibberella sp. F75 GAL36)
SEQ ID NO:5: an amino acid sequence of an alfa-galactosidase (Aspergillus fischeri GAL27B)
SEQ ID NO:6: an amino acid sequence of an alfa-galactosidase (S. cerevisiae MEL5)
SEQ ID NO:7: a polynucleotide sequence encoding an alfa-galactosidase (A. niger aglC)
SEQ ID NO:8: a polynucleotide sequence encoding an alfa-galactosidase (T. reesei agl1)
SEQ ID NO:9: a polynucleotide sequence encoding an alfa-galactosidase (Rhizomucor miehei GAL36)
SEQ ID NO:10: a polynucleotide sequence encoding an alfa-galactosidase (Gibberella sp. F75 GAL36)
SEQ ID NO:11: a polynucleotide sequence encoding an alfa-galactosidase (Aspergillus fischeri GAL27B)
SEQ ID NO:12: a polynucleotide sequence encoding an alfa-galactosidase (S. cerevisiae MEL5)
SEQ ID NO:13: primer 32 MEL5-ATG-F
SEQ ID NO:14: primer 33 MEL5-stopR
SEQ ID NO:15: a codon optimized polynucleotide sequence of a plasmid pMIE-16 (A. niger aglC; Q9UUZ4),
SEQ ID NO:16: a codon optimized polynucleotide sequence of a plasmid pMIE-17 (T. reesei agl1; Q92456)
SEQ ID NO:17: a codon optimized polynucleotide sequence of a plasmid pMIE-18 (Rhizomucor miehei GAL36; H8Y263)
SEQ ID NO:18: a codon optimized polynucleotide sequence of a plasmid pMIE-19 (Gibberella sp. F75 GAL36; C6FJG8)
SEQ ID NO:19: a codon optimized polynucleotide sequence of a plasmid pMIE-20 (Aspergillus fischeri GAL27B; AJA29661.1)
SEQ ID NO:20: a polynucleotide sequence of a plasmid pMIE-5 (S. cerevisiae MEL5)
SEQ ID NO:21: primer 2ScADH1-150F
SEQ ID NO:22: primer 5ScADH1 stopR
SEQ ID NO:23: a polynucleotide sequence of a plasmid pMIE-21B
SEQ ID NO:24: a polynucleotide sequence of a plasmid pMIE-24B
SEQ ID NO:25: a polynucleotide sequence of a plasmid pMIE-25B
SEQ ID NO:26: a polynucleotide sequence of a plasmid pMIE-26A
SEQ ID NO:27: a polynucleotide sequence of a plasmid pMIE-031
SEQ ID NO:28: a polynucleotide sequence of a plasmid pMIE-032
SEQ ID NO:29: a polynucleotide sequence of a plasmid pMIE-034
SEQ ID NO:30: primer 3ScPDC5-210F
SEQ ID NO:31: primer 6ScPDC5 stopR
SEQ ID NO:32: primer 4ScPDC5-136F
SEQ ID NO:33: a polynucleotide sequence of a plasmid pMIE-8
SEQ ID NO:34 an amino acid sequence of an invertase (S. cerevisiae SUC2)
SEQ ID NO:35 a polynucleotide sequence encoding an invertase (S. cerevisiae SUC2)
SEQ ID NO:36 a polynucleotide sequence of a plasmid pMIPk124
SEQ ID NO:37 a polynucleotide sequence of a plasmid pEKOPA8
SEQ ID NO:38 a polynucleotide sequence of a plasmid pEKOPA9

### DETAILED DESCRIPTION OF THE INVENTION

The object of the present invention has been achieved by increasing lactate dehydrogenase enzyme activity and alfa-galactosidase enzyme activity. The inventors of the present disclosure have been able to provide a fungus that has been genetically modified to increase lactate dehydrogenase enzyme and alfa-galactosidase enzyme activities.

In a method of the present invention for producing lactic acid and/or lactate, a fungus that has been genetically modified to increase lactate dehydrogenase enzyme and alfa-galactosidase enzyme activities is cultured in a medium comprising a carbon substrate to obtain said lactic acid and/or lactate.

As used herein “lactic acid” refers to an organic acid having a molecular formula CH₃CH(OH)CO₂H (chemical formula C₃H₆O₃). In industry lactic acid fermentation is performed by micro-organisms converting carbon substrates (e.g. simple carbohydrates such as glucose, sucrose or galactose) to lactic acid.

The lactic acid occurs in two stereoisomeric forms, D and L lactic acid, and in a so-called racemic mixture of these isomers. In one embodiment the lactic acid produced by the method or genetically modified fungus of the present invention is L-lactic acid isomer or D-lactic acid isomer or a combination thereof. In one embodiment the lactic acid is optically pure lactic acid isomer, optionally L-lactic acid isomer. As used herein “optically pure lactic acid isomer” refers to a solution or solid comprising substantially only one stereoisomeric form of lactic acid and not its mirror image (e.g. about 95% or more, about 96% or more, about 97% or more, about 98% or more, or about 99% or more (e.g. 99.5% or more) of one stereoisomeric form of lactic acid).

An effective fungus of the present invention was engineered to hydrolyze carbohydrates and convert them into lactic acid, e.g. into optically pure L-lactic acid. Said fungus was utilized in the method for producing lactic acid or lactate by culturing the fungus in a medium comprising a carbon substrate e.g. a carbon substrate comprising a simple and/or complex carbohydrate. Indeed, the present invention enables manipulation and control of a carbon source during large-scale production processes, which provides manufacturers with flexibility and excellent control over said processes. As used herein “a simple carbohydrate” refers to a simple sugar, which can be categorized as a single sugar (a monosaccharide), which comprises glucose, fructose and galactose, or a double sugar (a disaccharide), which comprises sucrose, lactose and maltose. As used herein “a complex carbohydrate” refers to a polysaccharide comprising three or more linked sugars. Indeed, it takes longer to break down a polysaccharide than a shorter non-polysaccharide.

Surprisingly, in one embodiment the fungus and method of the present invention are able to utilize complex carbohydrates, e.g. soy molasses, as a carbon substrate. In a specific embodiment of the invention, the carbon substrate comprises complex carbohydrates or is a complex carbohydrate. In a more specific embodiment, the carbon substrate comprises galacto-oligosaccharides or is a galacto-oligosaccharide. The most common galacto-oligosaccharides found in plant materials are the raffinose family oligosaccharides (RFOs). These molecules are derivatives of sucrose, with additional α-(1→6)-linked galactosyl moieties. The different RFO sugars according to the number of linked galactosyl units include raffinose (one galactose unit), stachyose (two galactose units), verbascose (three galactose units) and ajucose (four galactose units). In addition to RFOs, e.g. legumes may contain other galacto-oligosaccharides that contain terminal inositol groups, such as those belonging to the galactinol, galactopinitol and fagopyritol series of carbohydrates. In one embodiment of the invention the carbon substrate comprises complex carbohydrates or galacto-oligosaccharides at least about 10%, 20%, 30%, 40%, 50%, 60%, 70%, 80%, or 90% by weight of the total carbohydrates in said carbon substrate, and/or simple carbohydrates (e.g. glucose, fructose, galactose, sucrose, lactose or maltose or any combination thereof) at least about 10%, 20%, 30%, 40%, 50%, 60%, 70%, 80% or 90% by weight of the total carbohydrates in said carbon substrate.

In one embodiment of the invention the carbon substrate comprises a galacto-oligosaccharide or galacto-oligosaccharides, which is/are selected from the group consisting of melibiose, manninotriose, manninotetraose, raffinose, stachyose, verbascose, ajucose, galactinol, digalactosyl myo-inositol, galactopinitol A, galactopinitol B, ciceritol, fagopyritol B1, fagopyritol B2 and any combination thereof. In a specific embodiment the galacto-oligosaccharides are one or several from the group consisting of raffinose, stachyose, verbascose, melibiose, manninotriose and manninotetraose.

In one embodiment the carbon substrate comprises glucose, fructose, galactose, sucrose, lactose, maltose, starch, cellulose and/or any combination thereof. As used herein “starch” refers to a polymeric carbohydrate having the formula (C₆H₁₀O₅)ₙ—(H₂O), i.e. comprising or consisting of a large number of glucose units joined by glycosidic bonds. As used herein “cellulose” refers to an organic compound with the formula (C₆H₁₀O₅)ₙ, a polysaccharide consisting of a linear chain of several (e.g. from a hundred to many thousands) β(1-4) linked D-glucose units.

The carbon substrate used in the present invention may be obtained or may be from any carbon containing material, e.g. a combination of different carbon containing materials. In one embodiment the carbon substrate is from legumes such as soya (e.g. a soya bean), fava bean, peas, chickpeas, corn (e.g. a kernel of a corn cob), sugarcane (e.g. a plant), sugar beets (a beet of a sugar beet), lignocellulose or any combination thereof; and/or the carbon substrate comprises soy molasses, sugarcane molasses, sugar beet molasses and/or citrus molasses. As used herein “lignocellulose” refers to a material comprising cellulose, hemicelluloses and lignin. “Molasses” of e.g. soya, sugarcane, sugar beet or citrus refers to a product resulting from refining a bean, plant, beet or fruit, respectively, into sugar.

In one embodiment the carbon substrate or the medium, wherein the fungus is cultured, for producing lactic acid and/or lactate comprises 5-100 wt % soy molasses (e.g. at least about 5 wt %, 10 wt %, 20 wt %, 30 wt %, 40 wt %, 50 wt %, 60 wt %, 70 wt %, 80 wt %, or 90 wt %).

As an example, soy molasses is a side product of soy protein concentrate production. This is a low value stream that is normally destined to animal feed production or even burned. However, it may contain a very high concentration of soy carbohydrates (e.g. >300 g/L) that could be valorized. The challenge is that the sugars are nonconventional oligosaccharides such as raffinose and stachyose that need to be hydrolyzed and then all the resulting monosaccharides glucose, fructose and galactose need to be metabolized into a product. Soy molasses is an example of a cheaper feedstock for lactic acid production compared to e.g. corn starch and sucrose. Soy molasses can be used as a carbon substrate as such for fungal lactic acid production; there are no additional nutrient requirements, which further helps to minimize production costs of lactic acid.

To produce lactic acid the genetically modified fungus is cultured in a medium comprising an appropriate carbon source or sources and optionally other ingredients selected from the group consisting of nitrogen or a source of nitrogen (such as amino acids, proteins, inorganic nitrogen sources such as ammonia or ammonium salts), yeast extract, peptone, minerals and vitamins. In one embodiment, culturing of the fungus is carried out in suitable conditions known to a person skilled in the art. Suitable cultivation conditions, such as a temperature, pH, cell density, selection of nutrients, and the like are within the knowledge of a skilled person and said skilled person is able to choose, modify or control said conditions. In a specific embodiment the cultivation temperature is from about 25 to 45° C. (e.g. about 30-35° C.) and/or the pH of the medium is 2-10 (e.g. 3-6). Naturally, suitable cultivation conditions may depend on the specific fungus. The culturing conditions can be maintained during the method of producing lactic acid or lactate or alternatively, they can be adjusted periodically. In one embodiment, the culture conditions may vary in different tanks when more than one tank are used in the method for producing lactic acid or lactate.

In one embodiment of the invention the lactic acid or lactate is produced by an anaerobic, quasi-anaerobic or aerobic fermentation.

In one embodiment culturing of the fungus is carried out as a continuous fermentation method or as a batch or fed-batch fermentation method.

In one embodiment of the invention after culturing the genetically modified fungus in a medium, the method further comprises recovering the resulting lactic acid or lactate from the medium. Indeed, recovering can be carried out from the medium without disrupting the cells. In one embodiment after culturing the fungus in a medium, the method further comprises isolating and/or purifying lactic acid or lactate. Any suitable method known to a person skilled in the art can be used to isolate lactic acid or lactate. For example, common separation techniques can be used to remove the biomass from the medium, and common isolation procedures can be used to obtain lactic acid or lactate from the fungal-free media. Lactic acid or lactate can be isolated while it is being produced, or it can be isolated from the media after the lactic acid or lactate production has been terminated. Lactic acid and lactate can be recovered, isolated and/or purified by using any conventional methods known in the art such as adsorption, ion exchange procedures, chromatographic methods, two phase extraction, molecular distillation, melt crystallization, extraction, distillation or any combination thereof.

In one embodiment the fungus used during the production method is recovered and reused in subsequent production methods.

PLA, a thermoplastic aliphatic polyester, can be prepared from lactic acid, e.g. from the lactic acid produced and optionally recovered, isolated and/or purified by the method of present invention, by different methods including but not limited to the following: the ring-opening polymerization of lactide (derived from lactic acid) with various metal catalysts, direct condensation of lactic acid monomers, polymerization of lactic acid, contacting lactic acid with a zeolite, direct biosynthesis of PLA from lactic acid. In one embodiment the method of the present invention comprises preparing PLA from the obtained lactic acid.

The present invention relates to genetically modified yeasts and methods and uses related thereto, wherein the yeast has increased lactate dehydrogenase enzyme and alfa-galactosidase enzyme activities. The genetic modification utilized in the present invention is at least for modifying, more specifically increasing, activities of a lactate dehydrogenase and alfa-galactosidase. A lactate dehydrogenase allows production of lactic acid and lactate and an α-galactosidase enables degradation and consumption of complex carbohydrates including but not limited to soy molasses carbohydrates.

As used herein “lactate dehydrogenase enzyme activity” refers to an ability to catalyze conversion of pyruvate to lactate. Accordingly, “lactate dehydrogenase enzyme” refers to a protein having activity to convert pyruvate to lactate. An L-lactate dehydrogenase (L-LDH) enzyme converts pyruvate to L-lactate and a D-lactate dehydrogenase (D-LDH) enzyme converts pyruvate to D-lactate. L-lactate dehydrogenase and D-lactate dehydrogenase are classified as EC 1.1.1.27 and EC 1.1.1.28, respectively. Lactate dehydrogenase (LDH) refers to not only fungal or bacterial (such as Rhizopus oryzae or Lactobacillus helveticus) but also to any other LDH homologue from any micro-organism, organism or mammal, e.g. a bovine. Also, all isozymes, isoforms and variants are included with the scope of LDH. In a specific embodiment, the LDH is an L-LDH. The LDH protein and ldh gene of the R. oryzae ldhA (AF226154) and ldhB (AF226155) are identified in the article of Skory (2000 Appl. Environ. Microbiol. 66:2343-2348) and the L. helveticus ldhL (U07604) is identified in the article of Savijoki K., Palva A. (1997. Appl. Environ. Microbiol. 63:2850-2856), respectively. Examples of suitable open reading frames (ORF) include but are not limited to ORF of R. oryzae ldhA (Q9P4B6) and ldhB (Q9P4B5) and L. helveticus ldhL (CAB03618). As an example, ldh1, ldh2, ldh3, ldh4, ldh5, ldh5A, ldh6B, ldhA, ldhB, ldhC and ldhL encode related but not identical polypeptides, which are within the scope of ldh. The number of genes encoding related but not identical polypeptides depends on the micro-organism or organism in question.

As used herein “alfa-galactosidase enzyme activity” refers to an ability to catalyse the hydrolysis of the non-reducing terminal α-galactosyl residues from various α-galactosides, including galactose and raffinose oligosaccharides, galactomannans and galactolipids. Accordingly, “alfa-galactosidase enzyme” refers to a protein having activity to hydrolyze the non-reducing terminal α-galactosyl residues from various α-galactosides. Alfa-galactosidase is classified as EC 3.2.1.22. Alfa-galactosidase refers to not only fungal (such as S. cerevisiae) or bacterial but also to any other alfa-galactosidase homologue from any micro-organism or organism. Also, all isozymes, isoforms and variants are included with the scope of alfa-galactosidase. As an example (e.g. T. reesei) agl1, agl2 and agl3, (e.g. Aspergillus niger) aglA, aglB, aglC and aglD, and (e.g. S. cerevisiae) MEL1, MEL2, MEL5, and MEL6 encode related but not identical polypeptides, which are within the scope of alfa-galactosidase. The number of genes encoding related but not identical polypeptides depends on the micro-organism or organism in question.

An engineered fungus of the present invention comprises a genetic modification increasing protein or enzyme activity. As used herein, “increased protein or enzyme activity” refers to the presence of higher activity of a protein compared to a wild type protein, or higher total protein activity of a cell or fungus compared to an unmodified cell or fungus. Increased protein activity may result from up-regulation of the polypeptide expression, up-regulation of the gene expression, addition of at least part of a gene (including addition of gene copies or addition of a gene normally absent in said cell or fungus), increase of proteins and/or increased activity of a protein. Specific examples of generating increased protein or enzyme activities are provided in the Example section.

The presence, absence or amount of protein activities in a cell or fungus can be detected by any suitable method known in the art. Non-limiting examples of suitable detection methods include commercial kits on market, enzymatic assays, immunological detection methods (e.g., antibodies specific for said proteins), PCR based assays (e.g., qPCR, RT-PCR), and any combination thereof. In one specific embodiment the activity of the lactate dehydrogenase enzyme is determined by monitoring the absorbance after incubating the enzyme or fungus in the presence of lithium lactate and NAD+ e.g. as described in Tokuhiro et al. (2009, Appl Microbiol Biotechnol 82, 883-890) and/or the activity of the alfa-galactosidase enzyme is determined by measuring released p-nitrophenyl (pNP) after incubating the enzyme or fungus with p-nitrophenyl-α-galactopyranoside (pNPG) e.g. as described in Chen et al. (2015, Protein Expression and purification, 110, 107-114) and/or by measuring released methylumbelliferyl (MU) after incubating the enzyme or fungus with methylumbelliferyl-α-D-galactopyranoside (MUG) e.g. as described in Similä et al. (2010, J Microbiol Biotechnol, 20(12), 1653-1663).

Genetic modifications resulting in increased protein activity include but are not limited to genetic insertions, deletions or disruptions of one or more genes or a fragment(s) thereof or insertions, deletions, disruptions or substitutions of one or more nucleotides, or addition of plasmids. As used herein “disruption” refers to insertion of one or several nucleotides into the gene or polynucleotide sequence resulting in lack of the corresponding protein or presence of non-functional proteins or protein with lowered activity.

As used herein “up-regulation of the gene or polypeptide expression” refers to excessive expression of a gene or polypeptide by producing more products (e.g. mRNA or protein, respectively) than an unmodified fungus. For example one or more copies of a gene or genes may be transformed to a cell for upregulated gene expression. The term also encompasses embodiments, where a regulating region such as a promoter or promoter region has been modified or changed or a regulating region (e.g. a promoter) not naturally present in the fungus has been inserted to allow the over-expression of a gene. Also, epigenetic modifications such as reducing DNA methylation or histone modifications are included in “genetic modifications” resulting in upregulated expression of a gene or polypeptide. As used herein “increased or up-regulated expression” refers to increased expression of the gene or polypeptide of interest compared to a wild type fungus without the genetic modification. Expression or increased expression can be proved for example by western, northern or southern blotting or quantitative PCR or any other suitable method known to a person skilled in the art.

In certain embodiments, the engineered fungus comprises at least one (e.g. one, two, three, four, five, six or more) heterologous polynucleotide. Any of the inserted polynucleotides or genes (e.g. one, two, three, four, five, six or more) may be heterologous or homologous to the host fungus. The fungus can be genetically modified by transforming it with a heterologous polynucleotide that encodes a heterologous protein. Alternatively, for example heterologous promoters or other regulating sequences can be utilized in the fungus of the invention. As used herein “heterologous polynucleotide” refers to a polynucleotide not naturally occurring in a cell or fungus, i.e. a cell or fungus does not normally comprise said polynucleotide. Typically said heterologous polynucleotide has been inserted or modified by recombinant technology.

On the other hand, any of the inserted polynucleotides or genes (e.g. one, two, three, four, five, six or more) may be identical or very homologous to a fungus to be genetically modified. In that way e.g. the copy number of the polynucleotides or genes may be increased in the fungus compared to a genetically unmodified fungus. Alternatively, for example promoters or other regulating sequences identical or very homologous to the fungus to be genetically modified can be utilized. Indeed, the fungus of the present invention may be modified with a polynucleotide, which is normally comprised in said fungus, depending on the fungus in question.

In a specific embodiment the fungus that has been genetically modified does not originally (i.e. before said genetic modification) comprise a ldh gene (e.g. a L-ldh gene) and/or an alfa-galactosidase gene.

In one embodiment of the method, use or genetically modified fungus of the invention the alfa-galactosidase enzyme is a heterologous alfa-galactosidase enzyme and/or the lactate dehydrogenase enzyme is a heterologous lactate dehydrogenase enzyme.

If a heterologous alfa-galactosidase enzyme is utilized in the present invention, it can be an alfa-galactosidase from any suitable organism. In such a case, said heterologous alfa-galactosidase enzyme must be functional in the present invention. In one embodiment the heterologous alfa-galactosidase enzyme is an alfa-galactosidase enzyme of a yeast or filamentous fungus, e.g. selected from the genera Aspergillus, Gibberella, Cunninghamella, Fusarium, Glomus, Humicola, Mortierella, Mucor, Penicillium, Pythium, Rhizomucor, Rhizopus, Trichoderma and Saccharomyces, specifically from the group consisting of Gibberella zeae, Gibberella intermedia, Gibberella moniliformis, Gibberella fujikuroi, Gibberella nygamai, Gibberella sp. F75, Fusarium sp. 2 F75, Fusarium oxysporum, Fusarium mangiferae, Fusarium proliferatum, Fusarium verticilloides, Aspergillus nidulans, Aspergillus oryzae, Aspergillus terreus, Aspergillus niger, Aspergillus fischeri, Rhizopus miehei, Rhizomucor miehei, Rhizopus oryzae, Trichoderma reesei, Trichoderma harzianum, Trichoderma longibrachiatum and Saccharomyces cerevisiae. In a specific embodiment the heterologous alfa-galactosidase enzyme is, or the alfa-galactosidase gene is a functional alfa-galactosidase gene that encodes a protein, which is, at least 60%, 70%, 80%, 85%, 86%, 87%, 88%, 89%, 90%, 95%, 96%, 97%, 98%, or 99% identical to that encoded by a alfa-galactosidase gene e.g. of any of the species Aspergillus niger, Gibberella sp. F75, Aspergillus fischeri, Trichoderma reesei, Saccharomyces cerevisiae, Rhizomucor miehei.

If a heterologous lactate dehydrogenase enzyme is utilized in the present invention, it can be a lactate dehydrogenase from any suitable organism, including mammals such as a bovine. In such a case said heterologous lactate dehydrogenase enzyme must be functional in the present invention. In a specific embodiment the heterologous lactate dehydrogenase enzyme is from an organism, mammal, micro-organism, fungus, or bacterium, e.g. optionally from a mammal such as Bos (e.g. Bos taurus), a fungus such as Kluyveromyces or Rhizopus (e.g. Kluyveromyces thermotolerans or Rhizopus oryzae), or from bacteria such as Lactobacillus (e.g. Lactobacillus helveticus or L. casei), Pediococcus (e.g. Pediococcus acidilactici) or Bacillus (e.g. Bacillus megaterium), or from a unicellular protozoan parasite e.g. Plasmodium (e.g. Plasmodium falciparum). Ina specific embodiment the heterologous lactate dehydrogenase enzyme is, or the ldh gene is a functional ldh gene that encodes a protein, which is, at least 40%, 50%, 60%, 70%, 80%, 85%, 86%, 87%, 88%, 89%, 90%, 95%, 96%, 97%, 98%, or 99% identical to that encoded by a L-ldh gene e.g. of any of the species Lactobacillus helveticus, L. casei, Kluyveromyces lactis, Bacillus megaterium, Pediococcus acidilactici, Bos taurus, Rhizopus oryzae or Plasmodium falciparum. Examples of specific D-ldh genes are those obtained from L. helveticus, L. johnsonii, L. bulgaricus, L. delbrueckiii, L. plantarum, L. pentosus and P. acidilactici. Functional genes that are identical to such L-ldh or D-ldh genes or which are at least 35%, 60%, 70% or 80% identical to such genes at the amino acid level are suitable. In a specific embodiment L-ldh gene is obtained from L. helveticus or one that is at least 35%, 60%, 70%, 80%, 85%, 90% or 95% identical to said gene. Another suitable L-ldh gene is obtained from B. megaterium or one that is at least 35%, 60%, 70%, 80%, 85%, 90% or 95% identical to said gene. A suitable D-ldh gene is obtained from L. helveticus or is at least 45%, 60%, 70%, 80%, 85%, 90% or 95% identical to said gene.

In one embodiment of the invention the heterologous ldh and/or alfa-galactosidase gene is/are integrated into the genome of the fungus cell. In a specific embodiment, the ldh and/or alfa-galactosidase gene is/are integrated at a locus of a native PDC gene. The heterologous ldh and/or alfa-galactosidase gene can be e.g. under the transcriptional control of a promoter that is either native or heterologous to the fungus cell. In one embodiment the method, use or fungus may utilize a transformation vector comprising a functional ldh and/or alfa-galactosidase gene operatively linked to a promoter sequence that is e.g. native to a fungus to be genetically modified. It is possible to use different heterologous ldh and/or alfa-galactosidase genes under the control of different types of promoters and/or terminators.

In one embodiment a transformed fungal cell may contain a single ldh gene and/or alfa-galactosidase gene, or multiple ldh and/or alfa-galactosidase genes, such as from 1-10 ldh and/or alfa-galactosidase genes, especially from 1-5 ldh and/or alfa-galactosidase genes. When the transformed cell contains multiple ldh and/or alfa-galactosidase genes, the individual genes may be copies of the same gene, or include copies of two or more different ldh and/or alfa-galactosidase genes. Multiple copies of the heterologous and/or endogenous ldh and/or alfa-galactosidase genes may be integrated at a single locus (so they are adjacent to each other), or at several loci within the fungal cell's genome. As an example, two copies of similar or different ldh genes and/or alfa-galactosidase genes can be integrated at homologous alleles of a diploid fungus.

Methods of identifying cells that contain a heterologous polynucleotide of interest are well known to those skilled in the art. Such methods include, without limitation, PCR and nucleic acid hybridization techniques such as Northern and Southern analysis. In some cases, immunohistochemistry and biochemical techniques can be used to determine if a cell contains a particular nucleic acid by detecting the expression of the encoded enzymatic polypeptide encoded by that particular nucleic acid molecule. For example, an antibody having specificity for an encoded enzyme can be used to determine whether or not a particular cell or fungus contains that encoded enzyme. Further, biochemical techniques can be used to determine if a cell contains a particular nucleic acid molecule encoding an enzymatic polypeptide by detecting an organic product produced as a result of the expression of the enzymatic polypeptide.

In one embodiment of the method, use or fungus of the invention, the fungus has been genetically modified to overexpress a gene encoding a lactate dehydrogenase and/or a gene encoding an alfa-galactosidase. “Overexpression of a gene” refers to an up-regulated expression of said gene due to a genetic modification when compared to a fungus without said modification. In a specific embodiment said modified fungus comprises one or more copies of a gene encoding a lactate dehydrogenase and/or a gene encoding an alfa-galactosidase.

In one embodiment of the method, use or fungus of the invention, the gene encoding a lactate dehydrogenase is selected from the group consisting of ldh1, ldh2, ldh3, ldh4, ldh5, ldh6A, ldh6B, ldhA, ldhB, ldhC and ldhL, and/or the gene encoding an alfa-galactosidase is selected from the group consisting of agl1, agl2, agl3, aglA, aglB, aglC aglD, MEL1, MEL2, MEL5, and MEL6.

In one embodiment, in addition to genetic modifications resulting in increased lactate dehydrogenase and alfa galactosidase enzyme activities, the fungus of the present invention may further comprise one or several genetic modifications. In one embodiment, the fungus has further been genetically modified to decrease ethanol production. In a specific embodiment the fungus has been genetically modified to decrease ethanol production by modifying or deleting at least part of a gene associated with ethanol production or by inactivating a gene associated with ethanol production. Optionally the gene or genes associated with ethanol production is/are selected from the group consisting of PDC1, PDC5, PDC6, ADH1, ADH2, ADH3, ADH4, and ADH5, and any combination thereof. In one specific embodiment PDC1 and ADH1 have been deleted or modified. In another specific embodiment PDC1 and PDC5 have been deleted or modified. In a very specific embodiment one or more alleles of PDC1; PDC1 and ADH1; PDC1 and PDC5; ADH1 and PDC5; or PDC5 have been deleted or modified.

As used herein PDC gene refers to a gene encoding a pyruvate decarboxylase, which catalyzes the degradation of pyruvate into acetaldehyde and carbon dioxide. At least PDC1, PDC5, and PDC6 encode different isozymes of a pyruvate decarboxylase. The pyruvate decarboxylase is classified as EC 4.1.1.1. All isozymes, isoforms and variants are included with the scope of PDC.

As used herein ADH refers to a gene encoding a alcohol dehydrogenase, which catalyzes the conversion of acetaldehyde to ethanol. Yeast and most bacteria ferment carbon substrates such as glucose to ethanol and CO2. Indeed, pyruvate resulting from glycolysis is converted to acetaldehyde and carbon dioxide, and the acetaldehyde is then reduced to ethanol by an alcohol dehydrogenase. At least ADH1, ADH2, ADH3, ADH4, and ADH5 encode different isozymes of an alcohol dehydrogenase. The alcohol dehydrogenase is classified as EC 1.1.1.1. All isozymes, isoforms and variants are included with the scope of ADH.

In one embodiment a gene or genes associated with ethanol production is/are or has/have been modified or at least partly deleted or inactivated. In another embodiment any other gene than one associated with ethanol production is or has been modified or at least partly deleted or inactivated. In one embodiment of the present invention the fungus comprises a genetic modification reducing protein or enzyme activity. “Reduced activity” refers to the presence of less activity, if any, in a specific protein or modified fungus compared to a wild type protein or fungus, respectively, or lower activity (if any) in a cell or fungus compared to an unmodified cell or fungus. Reduced activity may result from down regulation of the polypeptide expression, down regulation of the gene expression, lack of at least part of the gene, lack of protein and/or lowered activity of the protein. There are various genetic techniques for reducing the activity of a protein and said techniques are well-known to a person skilled in the art. These techniques make use of the nucleotide sequence of the gene or of the nucleotide sequence in the proximity of the gene.

In a specific embodiment of the invention one or more proteins are inactivated. As used herein “inactivation” refers to a situation wherein activity of a protein is totally inactivated i.e. a cell has no activity of a specific protein. The gene can be inactivated e.g. by preventing its expression or by mutation or deletion of the gene or part thereof. In one embodiment of the invention one or more genes or any fragment thereof has been deleted. In a specific embodiment the fungus has been genetically modified by deleting at least part of a gene. As used herein “part of a gene” refers to one or several nucleotides of the gene or any fragment thereof. For example gene knockout methods are suitable for deleting the nucleotide sequence that encodes a polypeptide having a specific activity, of any part thereof.

Deletion or modification of the PDC and/or ADH genes can be accomplished in a variety of ways, including but not limited to a homologous recombination, a disrupted genetic locus, an antisense molecule or a killer plasmid present in the cell e.g. for reducing the expression of the PDC and/or ADH gene.

In one embodiment of the method, use or fungus of the invention, the fungus further comprises a genetic modification of one or more genes selected from the group consisting of CYB2, GPD1, GPD2, GPP1, GPP2 and any combination thereof. CYB2 encodes an L-lactate:cytochrome c oxidoreductase that oxidizes lactate. GPD1, GPP1 and GPP2 are genes associated with glycerol biosynthesis. GPD1 codes for a glycerol-3-phosphate dehydrogenase. GPP1 and GPP2 encode glycerol-1-phosphate phosphohydrolases 1 and 2, respectively.

The genetically modified fungi of the invention are obtained by performing specific genetic modifications. In one embodiment the genetically modified fungus is a recombinant fungus. As used herein, a “recombinant fungus” refers to any fungus that has been genetically modified to contain different genetic material compared to the fungus before modification (e.g. comprise a deletion, substitution, disruption or insertion of one or more nucleic acids including an entire gene(s) or parts thereof compared to the fungus before modification). “The recombinant fungus” also refers to a host cell comprising said genetic modification.

Polynucleotides encoding known polypeptides can be mutated using common molecular or genetic techniques. Nucleic acid and amino acid databases (e.g., GenBank) can be used to identify a polynucleotide sequence that encodes a polypeptide having enzymatic activity. Sequence alignment software such as BLAST (protein or nucleotide) can be used to compare various sequences. Briefly, any amino acid sequence having some homology to a polypeptide having enzymatic activity, or any nucleic acid sequence having some homology to a sequence encoding a polypeptide having enzymatic activity can be used as a query to search e.g. GenBank. Percent identity of sequences can conveniently be computed using BLAST software with default parameters. Sequences having an identities score and a positives score of a given percentage, using the BLAST algorithm with default parameters, are considered to be that percent identical or homologous.

In a specific embodiment of the invention a polypeptide used in the present invention comprises a sequence having a sequence identity of at least 30%, 35%, 40%, 45%, 50%, 55%, 60%, 65%, 70%, 75%, 80%, 85%, 90%, 95%, 96%, 97%, 98%, 99% or 100% to SEQ ID NO: 1, 2, 3, 4, 5, or 6, or an enzymatically active fragment or variant thereof. Sequences ID NO 1-6 are polypeptide sequences of alfa-galactosidases. In a specific embodiment of the invention a polynucleotide used in the present invention comprises a sequence having a sequence identity of at least 30%, 35%, 40%, 45%, 50%, 55%, 60%, 65%, 70%, 75%, 80%, 85%, 90%, 95%, 96%, 97%, 98%, 99% or 100% to SEQ ID NO: 7, 8, 9, 10, 11 or 12, or an active fragment or variant thereof. Sequences ID NO 7-12 are nucleotide sequences of alfa-galactosidase genes.

It is well known that a deletion, addition or substitution of one or a few amino acids does not necessarily change the catalytic properties of an enzyme protein. Therefore the invention also encompasses variants and fragments of the given amino acid sequences having the stipulated enzyme activity. The term “variant” as used herein refers to a sequence having minor changes in the amino acid sequence as compared to a given sequence. Such a variant may occur naturally e.g. as an allelic variant within the same strain, species or genus, or it may be generated by mutagenesis or other gene modification. It may comprise amino acid substitutions, deletions or insertions, but it still functions in substantially the same manner as the given enzymes, in particular it retains its catalytic function as an enzyme.

A “fragment” of a given protein or polypeptide sequence means part of that sequence, e.g. a sequence that has been truncated at the N- and/or C-terminal end. It may for example be the mature part of a protein comprising a signal sequence, or it may be only an enzymatically active fragment of the mature protein.

The present invention is based on a fungus and methods and uses related thereto. A variety of fungus are suitable for use in the present invention. In one embodiment the fungus is a yeast or filamentous fungus. In a specific embodiment the fungus is a yeast or filamentous fungus selected from the genera Aspergillus, Saccharomyces, Kluyveromyces, Pichia, Hansenula, Candida, Trichosporon, Rhizopus, Torulaspora, Issatchenkia and Scheffersomyces, e.g. specifically from the group consisting of Saccharomyces cerevisiae, S. uvarum, Kluyveromyces thermotolerans, K. lactis, K. marxianus, Hansenula polymorpha, Scheffersomyces stipitis, Rhizopus oryzae, Torulaspora pretoriensis, Issatchenkia orientalis, Pichia fermentans, P. galeiformis, P. deserticola, P. membranifaciens, P. jadinii, P. kudriavzevii, P. anomala, Candida ethanolica, C. sonorensis and C. apicola.

In one embodiment of the method, use or fungus of the present invention, the fungus has been deposited to the VTT Collection under the accession number VTT C-191026 or VTT C-201040. The following strain depositions according to the Budapest Treaty on the International Recognition of Deposit of Microorganisms for the Purposes of Patent Procedure were made at the VTT Culture Collection, P.O. Box 1000 (Vuorimiehentie 3), FI-02044 VTT, Finland: accession number VTT C191026 and accession number VTT C-201040. (For VTT C-191026 see E143-4 of example 3; for VTT C-201040 see example 6.)

The genetically modified fungus of the present invention can be prepared by any genetic method known to a skilled person. Said method comprises at least providing a fungus and genetically modifying the fungus to increase lactate dehydrogenase enzyme and alfa-galactosidase enzyme activities. Genetic modification of a fungus or fungal cell is accomplished in one or more steps via the design and construction of appropriate vectors and transformation of the fungal cell with said vectors. Electroporation and/or chemical (such as calcium chloride- or lithium acetate-based) transformation methods can be used. Methods for transforming a fungal cell are within the knowledge of a skilled artisan. Examples of possible genetic modifications have been described above in the disclosure. In one embodiment one or more polynucleotides encoding one or more heterologous enzymes are added to the fungus or fungal cell, and optionally one or more polynucleotides encoding one or more endogenous enzymes are modified (e.g. by insertion, deletion or substitution of one or more nucleotides) to increase or decrease the activity of said enzymes in said fungus. The knowledge of a polynucleotide sequence encoding a polypeptide or a polypeptide sequence can be used for genetically modifying a suitable fungus.

The genetically modified fungus of the present invention is capable of hydrolysing the non-reducing terminal α-galactosyl residues from various α-galactosides, consuming pyruvate and producing lactic acid and/or lactate, when the fungus is present in a fermentation medium comprising galacto-oligosaccharides. In a very specific embodiment said fungus can produce L-lactic acid with high productivity and yield. In one embodiment the fungus of the present invention tolerates high lactic acid concentrations. In a very specific embodiment the fungus is an acid tolerant fungus modified for minimal production of native fermentation product ethanol and instead produce lactic acid.

In one embodiment of the invention the fungus has increased lactic acid production. The methods for producing lactic acid can result in lactic acid titers of about 30, 40, 50, 60, 70, 80, 90, 100, 110, 120, or 130 grams/L or more and/or lactic acid productivities of about 0.5, 1.0, 1.5, 2.0, 2.5, 3.0 g L⁻¹ h⁻¹ or more.

In one embodiment the fungus of the present invention has a very excellent performance, converting sugars (e.g. soy molasses sugars) at over 80% yield (i.e., g organic product/g carbon source consumed), over 2 g L⁻¹ h⁻¹ productivity and reaching high titers (up to 129 g/L lactic acid).

The methods for producing lactate can result in lactate titers of about 30, 40, 50, 60, 70, 80, 90, 100, 110, 120, or 130 grams/L or more, and/or lactate productivities of about 0.5, 1.0, 1.5, 2.0, 2.5, 3.0 g L⁻¹ h⁻¹ or more.

Methods of detecting lactic acid, lactate and/or galacto-oligosaccharides are well known to those skilled in the art. For example, chromatographic methods such as HPLC and ion chromatography can be used. The presence of lactate can be determined e.g. as described in Witte et al. (1989, J. Basic Microbiol. 29: 707-716).

The fungus of the present invention can be used for producing lactic acid and/or lactate or for producing polymers, optionally polyesters or polylactic acids.

A method of the present invention for producing one or more products selected from the group consisting of polymers, polyesters and polylactic acids, comprises culturing the genetically modified fungus of the present invention in a carbon substrate (e.g. galacto-oligosaccharides) containing medium to produce lactic acid, recovering the resulting lactic acid and utilizing the recovered lactic acid in production of polymers, polyesters and/or polylactic acids. Production of polymers is a well known method to a person skilled in the art including but not limited to e.g. polymerization of lactic acid.

In the present disclosure, the terms “polypeptide” and “protein” are used interchangeably to refer to polymers of amino acids of any length. As used herein “an enzyme” refers to a protein or polypeptide which is able to accelerate or catalyze chemical reactions.

As used herein “polynucleotide” refers to any polynucleotide, such as single or double-stranded DNA (genomic DNA or cDNA) or RNA, comprising a nucleic acid sequence encoding a polypeptide in question or a conservative sequence variant thereof. Conservative nucleotide sequence variants (i.e. nucleotide sequence modifications, which do not significantly alter biological properties of the encoded polypeptide) include variants arising from the degeneration of the genetic code and from silent mutations.

It will be obvious to a person skilled in the art that, as the technology advances, the inventive concept can be implemented in various ways. The invention and its embodiments are not limited to the examples described below but may vary within the scope of the claims.

### EXAMPLES

### Example 1—Growth of Different Fungal Species on Galactose

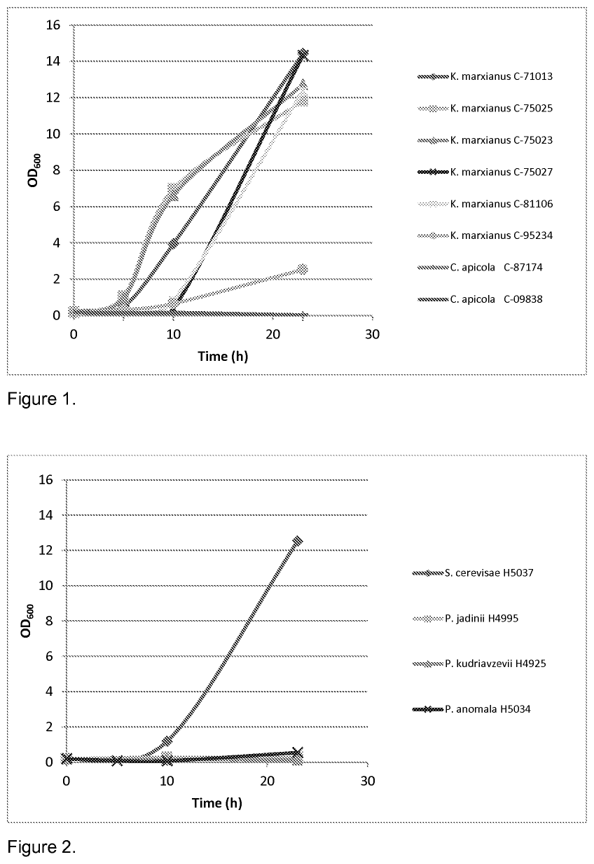

The growth of several wild-type and ldh-expressing strains of fungus on galactose was studied in shake flask cultivations. The strains were cultivated in 50 mL Erlenmeyer bottles with 10 mL of SC media, Yeast Nitrogen Base and 20 g/L of galactose as carbon source. The growth of the strains was evaluated by quantifying optical density (OD₆₀₀) during the course of the cultivations. Among the wild-type strains (FIG. 1) all Kluyveromyces marxianus strains were able to grow on galactose, while neither of the two tested Candida apicola strains showed demonstrable growth. Among the strains expressing L. helveticus ldhL coding for L-lactate dehydrogenase only Saccharomyces cerevisiae H5037 (derived from wild-type strain C-02453) grew well, while none of the strains belonging to genus Pichia, P. jadinii, P. kudriavzevii, or P. anomala, were able to grow on this sugar (FIG. 2). In conclusion, there is significant variation between fungal or yeast species in their ability to utilize galactose as a carbon source.

### Example 2—Demonstration of α-Galactosidase Activity in Fungus

S. cerevisiae strain VTT-C-02453 was received from VTT Culture Collection. All other strains are descendants of VTT-C-02453.

An uridin auxotrophic derivative of S. cerevisiae VTT-C-02453 was constructed by replacing protein coding region of the URA3 gene by the hph gene conferring hygromycin resistance. The hph expression cassette was flanked by loxP sites to facilitate marker excision by cre recombinase. Both URA3 alleles were deleted in the diploid host.

For multicopy episomal expression of α-galactosidase, the S. cerevisiae MEL5 gene (Genbank accession number Z37511) was amplified by PCR from plasmid pMLV18 (pMEL5-39 derivative, Naumov et al. 1990. Mol Gen Genet 224:119-128; Turakainen et al. 1994 Yeast 10:1559-1568) using primers 32 MEL5-ATG-F (SEQ ID NO: 13) and 33 MEL5-stopR (SEQ ID NO: 14), digested with EcoRI and Ascl, and cloned between S. cerevisiae ENO1 promoter and terminator into pMI529 (II-mén et al 2011 Biotech for Biofuels 4:30), resulting in pMIE-005. The protein coding regions of other α-galactosidase encoding genes were synthesized and optimized for expression in S. cerevisiae by Genscript (USA), and the MEL5 gene in pMIE-5 was replaced by the synthetic genes resulting in plasmids pMIE-16 (A. niger aglC; Q9UUZ4) (SEQ ID NO: 15), pMIE-17 (T. reesei agl1; Q92456) (SEQ ID NO: 16), pMIE-18 (Rhizomucor miehei GAL36; H8Y263) (SEQ ID NO: 17), pMIE-19 (Gibberella sp. F75 GAL36; C6FJG8) (SEQ ID NO: 18), and pMIE-20 (Aspergillus fischeri GAL27B; AJA29661.1) (SEQ ID NO: 19).

VTT-C-02453 ura3Δ/ura3Δ was transformed with each of the URA3 selectable α-galactosidase expression vectors pMIE-5 (S. cerevisiae MEL5) (SEQ ID NO: 20), pMIE-16 (A. niger aglC), pMIE-17 (T. reesei agl1), pMIE-18 (Rhizomucor miehei GAL36; H8Y263), pMIE-19 (Gibberella sp. F75 GAL36; C6FJG8), or pMIE-20 (Aspergillus fischeri GAL27B; AJA29661.1) using the lithium acetate method (Gietz et al. 1992 Nucleic Acids Res. 20:1425.). Transformants were selected on SCD-Ura medium. α-galactosidase activity was observed based on formation of blue colour of the colonies on agar plates supplemented with 5-bromo-4-chloro-3-indolyl-α-D-galactopyranoside (α-X-gal).

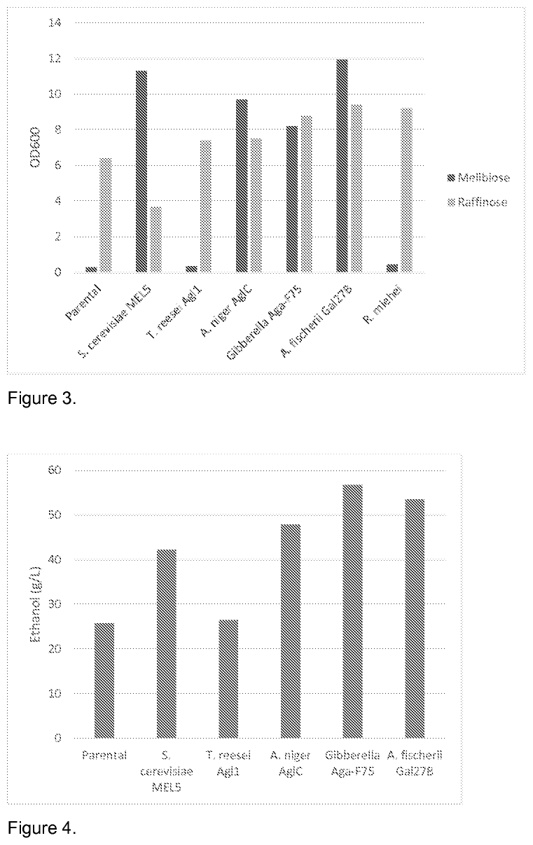

α-galactosidase genes activity on α-X-gal was observed in each of the yeast transformants expressing an α-galactosidase (data not shown). The ability of the α-X-gal positive transformants to grow in liquid SC-Ura-medium containing 1% melibiose or raffinose as the only carbon source was tested in 4 ml o/n cultures on 24-well plates at 30° C. at 220 rpm shaking. The parent strain containing a functional URA3 gene was included as a negative control. Transformants expressing α-galactosidases of S. cerevisiae, A. niger, Gibberella sp., or Aspergillus fischeri grew well on melibiose to OD₆₀₀ of 8 to 12, while the OD₆₀₀ of the parent strain lacking an α-galactosidase and transformants harbouring the T. reesei or R. miehei α-galactosidase genes had OD₆₀₀ below 1 (FIG. 3). In comparison, growth on raffinose is not solely dependent on α-galactosidase, since invertase cleaves raffinose to fructose and melibiose, and fructose can be consumed by the parent strain.

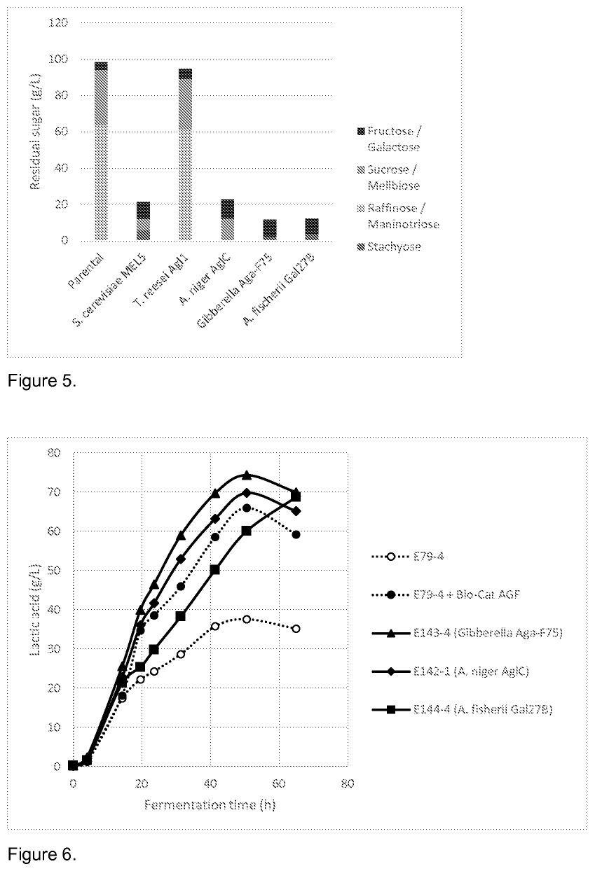

The pMIE-5 (S. cerevisiae MEL5), pMIE-16 (A. niger aglC), pMIE-17 (T. reesei agl1), pMIE-19 (Gibberella sp. F75 GAL36; C6FJG8), and pMIE-20 (Aspergillus fischeri GAL27B; AJA29661.1) transformants (see example 2) were cultivated for 24 hours in 1:3 diluted soy molasses in 4 ml on 24-well plates to demonstrate the ability of the strains to convert the different sugars to ethanol. Filtered samples were run on an Aminex HPX-87H column (Bio Rad), 35° C., 0.3 mL/min flow of 5 mM H2SO4 to quantify produced ethanol and residual sugars. The method does not distinguish trisaccharides (raffinose/manninotriose) or disaccharides (sucrose, melibiose), and does not separate fructose from galactose. Ethanol production was increased considerably relative to the parent strain VTT-C-02453 ura3Δ/ura3Δ when S. cerevisiae MEL5, A. niger aglC, Gibberella sp. F75 GAL36 or A. fischeri GAL27B was expressed (FIG. 4). The consumption of soy molasses galacto-oligosaccharides (GOS) by these strains was also evident from the HPLC results (FIG. 5). The parent strain and the strain expressing T. reesei AGL1 showed significant residual di- and tri-saccharides, while these were not evident for the strains expressing S. cerevisiae MEL5, A. niger aglC, Gibberella sp. F75 GAL36 or A. fischeri GAL27B.

### Example 3—Construction of Fungus Expressing LDH and Different α-galactosidases

ADH1 gene in VTT-C-02453 was deleted by replacing the coding region by a PCR product containing the KanMX geneticin resistance cassette, flanked by loxP sites, which was amplified from pUG6 (=B901) using primers 2ScADH1-150F (SEQ ID NO: 21) and 5ScADH1stopR (SEQ ID NO: 22) for the deletion construct 2+5-ScADH1.

For integration of the different α-galactosidase expression cassettes into the S. cerevisiae CAN1 locus, pMIE-5, pMIE-16, pMIE-19 pMIE-20 were digested with Smal and Swal, dephosphorylated, and the α-galactosidase containing fragments were ligated to the 5177 bp Mscl-EcoRV fragment of B3033=pMI-503 containing the KanMX cassette and CAN1 homology regions, resulting in pMIE-21B (SEQ ID NO: 23), pMIE-24B (SEQ ID NO: 24), pMIE-25B (SEQ ID NO: 25), pMIE-26A (SEQ ID NO: 26), respectively.

For integration of the Lactobacillus helveticus ldhL coding for L-lactate dehydrogenase into the PDC1 locus, the expression vector pMIE-8 (SEQ ID NO: 33) was constructed. It contains the L. helveticus ldhL between S. cerevisae PGK1 promoter and ADH1 terminator and the E. coli hph gene between A. gossypii TEF1 promoter and terminator conferring hygromycin resistance, surrounded by loxP sites for marker excision, and 5′ and 3′ regions of PDC1 facilitating homologous recombination into the PDC1 locus.

For marker excision the cre recombinase was expressed under the GAL1 promoter from a nourseothricin selectable centromeric vector cre-NAT.

S. cerevisiae was transformed using the PEG-lithium acetate method (Gietz et al. 1992 Nucleic Acids Res. 20:1425). Transformants were selected in agar-solidified YPD medium supplemented with 200 μg/ml hygromycin, 300 μg/ml geneticin, or 200 μg/ml nourseothricin, as appropriate.

VTT-C-02453 was transformed with pMIE-8 and a hygromycin resistant transformant E16 was isolated. The hygromycin resistance marker was excised by transforming a cre-recombinase expression vector pSK-70 into E16 and a nourseothricin-resistant transformant E23 was isolated. E23 was transformed with pMIE-8 and a hygromycin resistant transformant E51-6 was isolated. PCR analysis indicated that PDC1 coding region was absent from E51-6. E51-6 was transformed with the ADH1 deletion cassette and G418 resistant transformants E79-4, E79-5, E79-9 and E79-10 were isolated. PCR analysis indicated that an ADH1 coding region was present in E79-5, E79-9 and E79-10 but absent from E79-4 suggesting that both ADH1 alleles were deleted from E79-4. In accordance with this, E79-4 formed smaller colonies than E79-5, E79-9 and E79-10. The resistance markers were excised by transforming cre-recombinase expression vector pSK-70 into E79-4 and nourseothricin-resistant transformants were isolated.

Markerless derivative of transformant E79-4 was transformed with SacII-ScaI digested pMIE-24B, pMIE-25B, and pMIE-26A, for expression of α-galactosidase genes of A. niger, Gibberella sp., and A. fischeri, respectively. The α-galactosidase genes were targeted for integration into the CAN1 locus. Transformants were selected based on geneticin resistance. α-galactosidase activity was observed based on formation of blue colour of the colonies on agar plates supplemented α-X-gal. Strains E142-1, E143-4 (VTT C-191026) and E144-4 express the α-galactosidase genes of A. niger, Gibberella sp. F75 and A. fischeri, respectively.

S. cerevisiae strain E79-4 engineered from VTT-C-02453 for lactic acid production and reduced ethanol production (for ADH1 gene deletion and ldhL integration see example 2) was cultivated in bioreactors using soy molasses as the sole carbon source. The lactic acid production of this strain was compared to derived strains expressing different heterologous α-galactosidases integrated into the CAN1 locus as described in Example 2. In addition, the parental strain E79-4 was cultivated with an initial dose of 5 U/mL of commercial alpha-galactosidase (BioCat AGF). The strains were cultivated using an Infors Multifors bioreactor system. The batch medium comprised autoclaved soy molasses, diluted to one-sixth its original volume in reverse osmosis (RO) water, with 80 g/L CaCO₃ as a buffering agent and 1 mL/L Adeka nol 109 as antifoam agent. The used fermentation conditions were: Temperature—30° C., agitation—550 rpm, aeration—0.15 LPM. All strains were pre-cultivated in shake flasks on standard YPD medium for 2 days. The cells were centrifuged and washed twice with water before resuspending them in the fermentation batch medium prior to inoculation into the bioreactors. The initial pitch of cells was normalized to correspond to a starting optical density (OD₆₀₀) of 1. After 20 hours of fermentation, a total of 250 mL of autoclave-sterilized soy molasses diluted to one-third its original volume with RO-water was fed into the reactors at a rate of approximately 8 mL/h.

Samples were withdrawn from the reactors at regular intervals, and the produced lactic acid and residual carbohydrates were quantified. Lactic acid was quantified by HPLC using an Aminex HPX-87H column (Bio Rad), 35° C., 0.3 mL/min flow of 5 mM H₂SO₄. Galacto-oligosaccharides (GOS) were quantified using a Dionex ICS-3000 system and a CarboPac PA1 column. Total GOS are reported as the sum of the concentrations of raffinose, stachyose, verbascose, melibiose, manninotriose and manninotetraose.

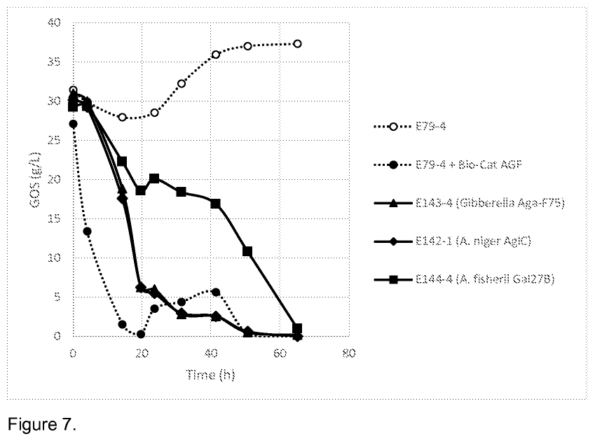

The results demonstrate a significant increase in lactic acid production, when the fungus was able to utilize raffinose family oligosaccharides as a carbon source through the action of α-galactosidase (FIG. 6). The degradation of galacto-oligosaccharides could be seen as a significant reduction of these sugars in the culture supernatants (FIG. 7). Surprisingly, the strains expressing α-galactosidase reached higher lactate titers than what was achieved using added commercial enzyme.

The expression level of α-galactosidase was further modified in E142-1 and E143-4 (VTT C-191026) expressing α-galactosidase A. niger or Gibberella sp. F75, respectively, by integration of a second of α-galactosidase gene into the remaining CAN1 allele. E142-1 and E143-4 (VTT C-191026) were transformed separately with KpnI-SapI digested pMIE-031 (SEQ ID NO: 27), pMIE-032 (SEQ ID NO: 28), and pMIE-034 (SEQ ID NO: 29) carrying A. niger aglC, Gibberella sp. F75 GAL36 and A. fischeri GAL27B genes, respectively. Transformants were selected based on hygromycin resistance. Transformants deleted of both CAN1 alleles express two copies of A. niger aglC (E157), A. niger aglC and Gibberella sp. F75 GAL36 (E158, E160), two copies of Gibberella sp. F75 GAL36 (E161) and Gibberella sp. F75 GAL36 and A. fischeri GAL27B (E162). Production of lactic acid is demonstrated in bioreactors using soy molasses as the sole carbon source as described above.

### Example 4—Production of Lactic Acid Using Fungus Expressing Ldh and Different α-Galactosidases

PDC5 gene was deleted by replacing the coding region by a PCR product containing the KanMX geneticin resistance cassette, flanked by loxP sites, which was amplified from pUG6 (=B901) using primers 3ScPDC5-210F (SEQ ID NO: 30 and 6ScPDC5stopR (SEQ ID NO: 31).

VTT-C-02453 was transformed with the above mentioned PDC5 deletion cassette and G418 resistant transformant E3 was isolated. E3 was transformed with NotI digested pMIE-8 and a hygromycin resistant transformant E15 was isolated. The KanMX and hygromycin resistance markers were excised by transforming a cre-recombinase expression vector pSK-70 into E15 and a nourseothricin-resistant transformant E22 was isolated.

E22 was transformed with pMIE-8 and a hygromycin resistant transformants were isolated. PCR analysis indicated that PDC1 coding region was absent from transformant E68-1. E68-1 is transformed with the PDC5 deletion cassette, which was prepared by PCR using primers 4ScPDC5-136F (SEQ ID NO: 32) and 6ScPDC5stopR (SEQ ID NO: 31) and the pUG6 plasmid as the template, and G418 resistant transformant E82 is isolated. The absence of PDC5 coding region in the transformants is verified with PCR.

In parallel, E22 was transformed with the PDC5 deletion cassette and G418 resistant were isolated. PCR analysis indicated that an PDC5 coding region was not present in transformant E78-1 suggesting that both PDC5 alleles were deleted from E78-1. E78-1 is transformed with NotI digested pMIE-008 in order to delete the remaining PDC1 allele and hygromycin resistant transformants are isolated. The absence of PDC1 coding region in the transformant E94 is verified by PCR.

The transformants E82 and E94, deleted of both copies of pdc1 and pdc5, are transformed with the cre-recombinase expression vector pSK-70 in order to excise the KanMX and hygromycin resistance markers. Markerless derivatives of transformants E82 and E94 are transformed with SacII-ScaI digested pMIE-24B, pMIE25B, and pMIE-26A, for expression of α-galactosidase genes of A. niger, Gibberella sp., and A. fischeri, respectively. The α-galactosidase genes were targeted for integration into the CAN1 locus. Transformants are selected based on geneticin resistance. α-galactosidase activity is observed based on formation of blue colour of the colonies on agar plates supplemented α-X-gal. Production of lactic acid is demonstrated in bioreactors using soy molasses as the sole carbon source as described in Example 3.

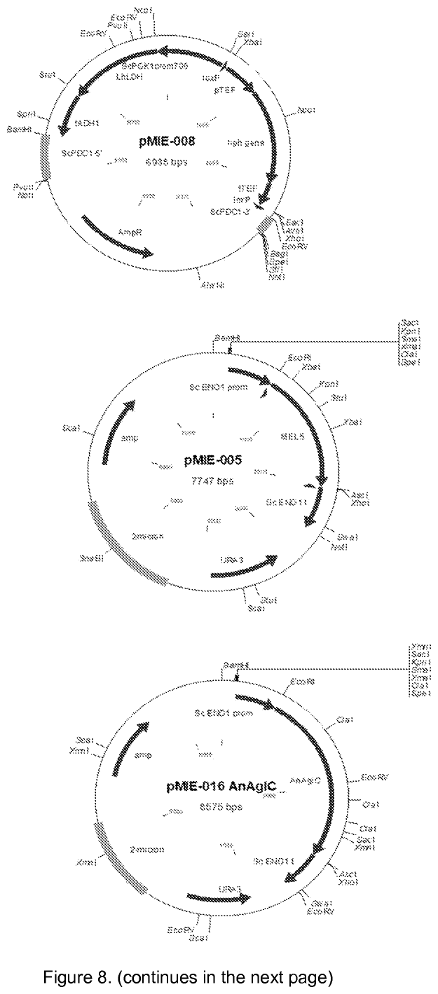

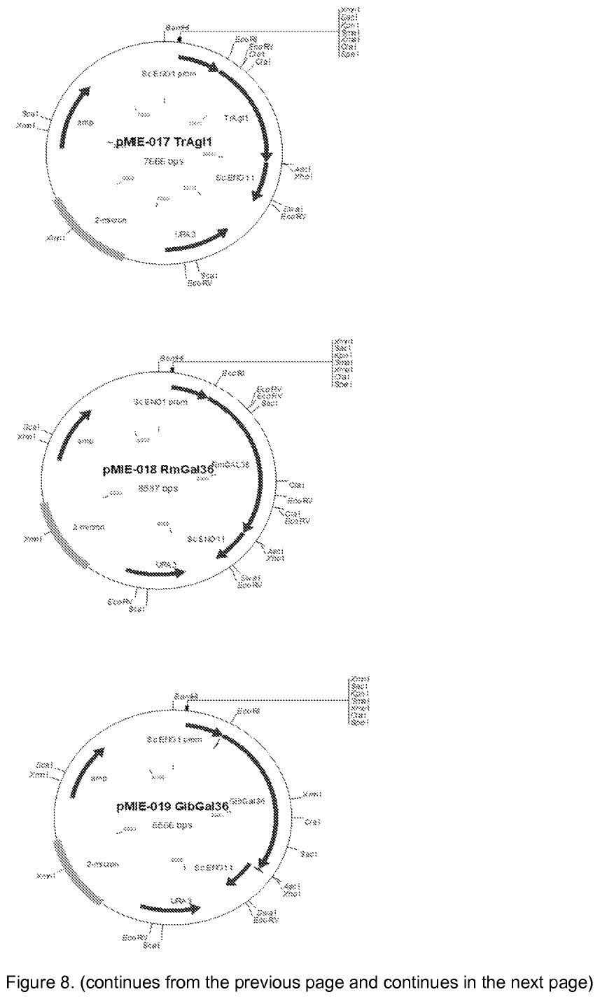

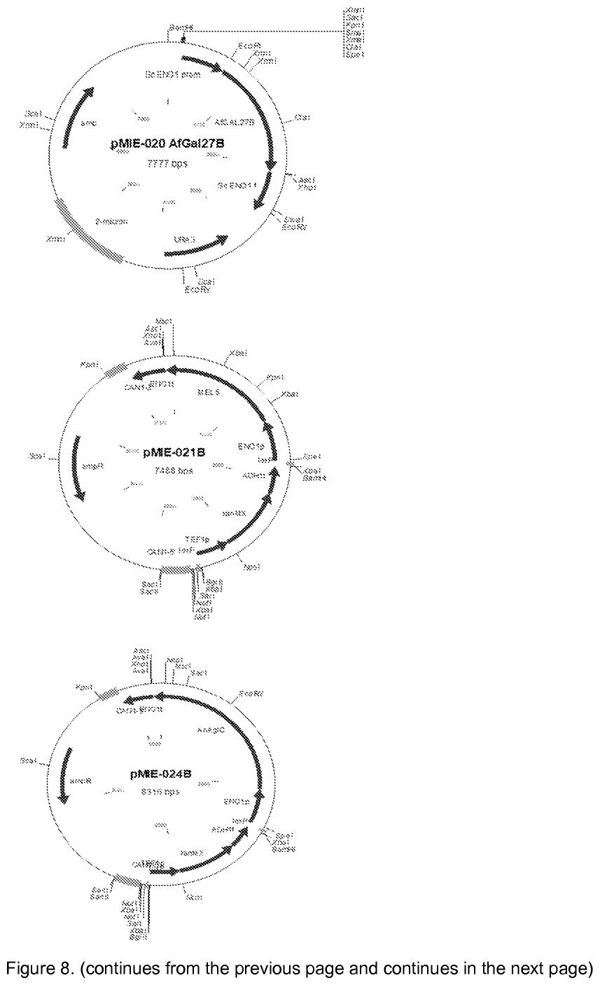

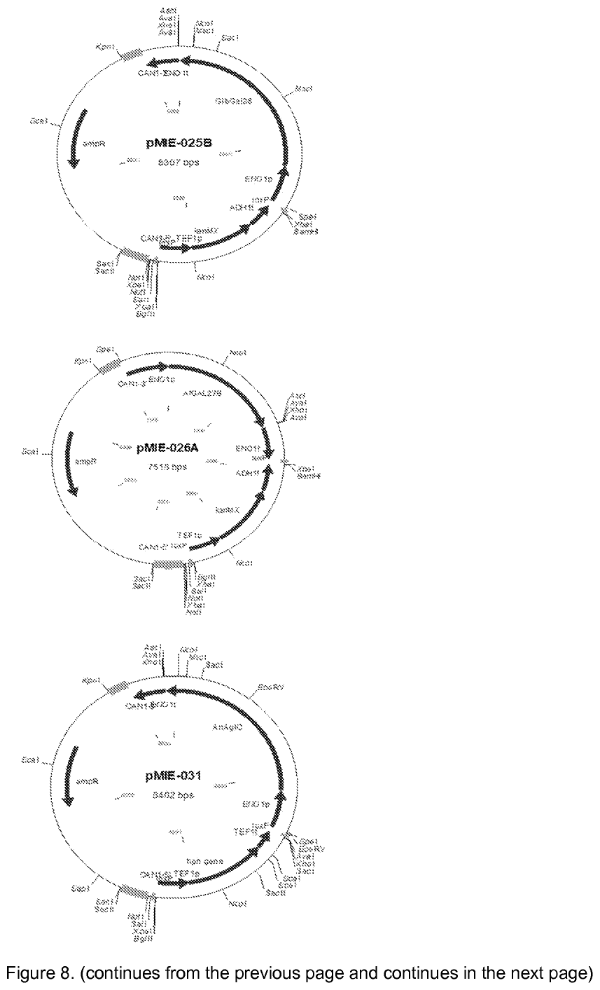

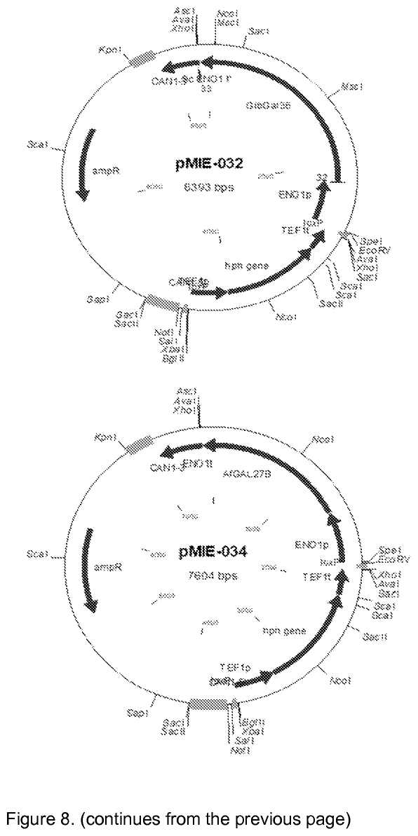

FIG. 8 shows maps of the plasmids described or mentioned in examples 1-4.

### Example 5—Lactate Production by Strains Expressing More than One α-Galactosidase

Strain VTT C-191026 (E143-4, see example 3) and three strains containing additional α-galactosidase genes were cultivated in shake flasks using soy molasses as carbon source. The three strains contained either an additional copy of Gibberella sp. F75 GAL36, or an A. niger agIC or a A. fischerii GAL27B as described in Example 3. Pre-cultures of the different strains were grown overnight in YPD medium at 30° C. The cells were harvested by centrifugation and resuspended in RO-H₂O to give an OD₆₀₀ value of 20. Soy molasses was diluted to one third its original concentration with RO-H₂O and sterilized using a standard autoclave liquid cycle (121° C., 20 min). 50 milliliters of this sterilized, diluted soy molasses were added to 250 mL Erlenmeyer flasks, which had been pre-sterilized with 2.5 g of CaCO₃ using a dry cycle (160° C., 3h). 500 microliters of cell suspension was used to inoculate each cultivation bottle, for an initial cell density corresponding to an OD₆₀₀ value of approximately 0.2.

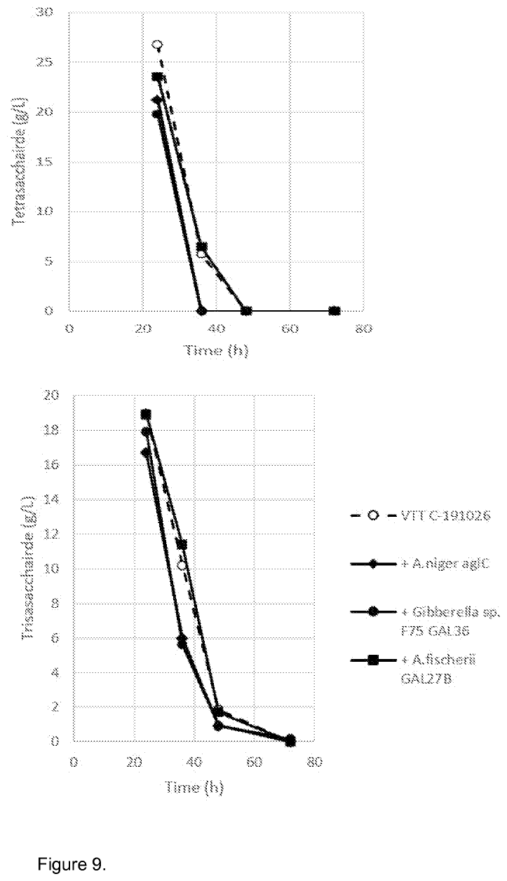

The flasks were maintained in a shaking incubator at 30° C. with 200 rpm agitation, and samples withdrawn periodically. The samples were centrifuged and the resulting supernatants immersed in a boiling water bath for 10 minutes. After boiling, the samples were centrifuged again, and the resulting supernatants diluted 10-fold in HPLC eluent (5 mM H₂SO₄). The samples were run on an Aminex HPX-84H column (Bio-Rad) at 55° C. and 0.5 mL flow rate. Stachyose was used as standard for tetrasaccharide, while maltotriose and maltose were used as standards for tri- and di-saccharides, respectively. The obtained results are given in FIG. 9 and suggest that additional copies of α-galactosidase genes could further enhance the rate of hydrolysis of soy molasses galacto-oligosaccharides compared to VTT C191026.

### Example 6—Production of Lactic Acid by Alternative Yeast P. Kudriavzevii

To demonstrate that expressing α-galactosidase and lactate dehydrogenase in yeasts other than S. cerevisiae could also result in high-level production of lactic acid from soy molasses, a suitable strain (VTT C-201040) was generated from Pichia kudriavzevii VTT-C-79090. As the yeast is naturally not able to hydrolyze sucrose, the additional expression on invertase was required.

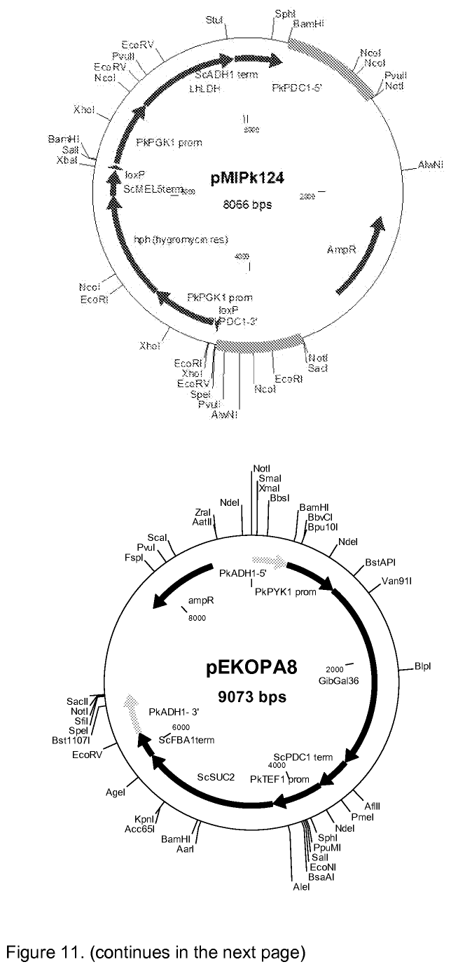

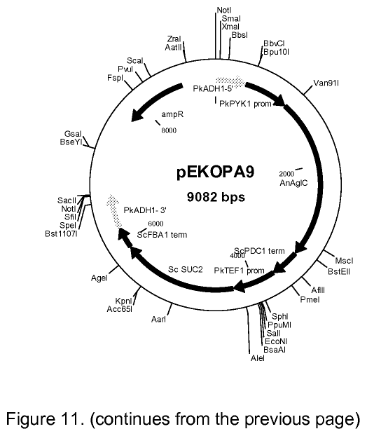

For integration of the L. helveticus ldhL coding for L-lactate dehydrogenase into the PDC1 locus, the expression vector pMIPk124 (SEQ ID NO: 36, FIG. 11) was constructed. It contains the L. helveticus ldhL between P. kudriavzevii PGK1 promoter and S. cerevisiae ADH1 terminator and the E. coli hph gene between P. kudriavzevii PGK1 promoter and S. cerevisiae MEL5 terminator conferring hygromycin resistance, surrounded by loxP sites for marker excision, and 5′ and 3′ regions of P. kudriavzevii PDC1 facilitating homologous recombination into the PDC1 locus. The expression cassettes were released from vector sequences by Notl digestion. P. kudriavzevii was transformed using the PEG-lithium acetate method (Gietz et al. 1992 Nucleic Acids Res. 20:1425). Transformants were selected in agar-solidified YPD medium supplemented with 500 μg/ml hygromycin or 200 μg/ml nourseothricin, as appropriate. The hygromycin resistance marker was excised from transformant H4868 by transforming a cre-recombinase expression vector pKLNatCreloPGK into and a nourseothricin-resistant transformant was isolated. pKLNatCreloPGK was removed by growing the cells on non-selective medium resulting in isolation of strain H4927. H4927 was transformed again with pMIPk124 to replace both PDC1 alleles in the diploid genome with the ldhL expression vector, and H4948 was isolated.

The hygromycin resistance marker was removed from the strain H4948 with cre-recombinase similarly as described above and the strain obtained was named H5661. H5661 was the parental strain for integration of invertase and alpha-galactosidase into the ADH1 locus. Two expression vectors pEKOPA8 (SEQ ID NO: 37, FIG. 11) and pEKOPA9 (SEQ ID NO: 38, FIG. 11) were constructed containing S. cerevisiae SUC2 (SEQ ID NO: 35) coding for invertase (SEQ ID NO: 34) together with either Gibberella GibGAL36 (pEKOPA8) or Aspergillus niger AgIC (pEKOPA9) each coding for an α-galactosidase, and 5′ and 3′ regions of P. kudriavzevii ADH1 facilitating homologous recombination into the ADH1 locus. The double expression cassettes were released from the vectors for transformation with Notl restriction enzyme. Transformants expressing invertase and alpha-galactosidase were selected in agar-solidified YP medium supplemented with 20 g/l D(+)-sucrose and 40 μg/ml α-X-Gal.

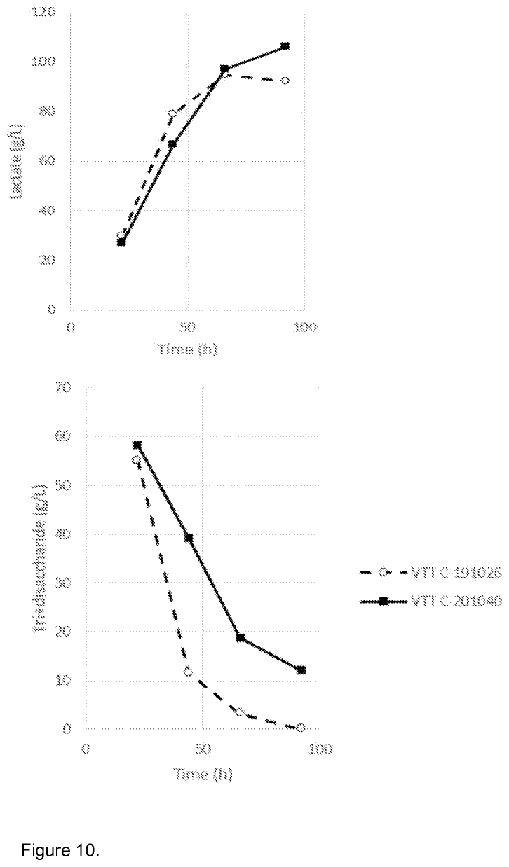

To demonstrate lactic acid production from soy molasses, the P. kudriavzevii strain VTT-C-201040 expressing invertase and Gibberella GibGAL36 alpha-galactosidase was cultivated in shake flasks using soy molasses as carbon source in parallel with VTT C-191026. The cultivation conditions were the same as described in Example 5. Produced lactic acid and residual oligosaccharides were quantified from culture samples as described in previous examples, and results are given in FIG. 10. Comparable levels of lactic acid production was achieved with both strains. The results indicate that high levels of lactic acid production from soy molasses could be achieved using another yeast strain with similar genetic modifications.

FIG. 11 shows maps of the plasmids described or mentioned in example 6.

1. A method of producing lactic acid and/or lactate, said method comprising:
providing a genetically modified fungus overexpressing a gene encoding a lactate dehydrogenase and a gene encoding an alfa-galactosidase;
culturing said fungus in a medium comprising a carbon substrate comprising galacto-oligosaccharides to obtain lactic acid and/or lactate, wherein the carbon substrate comprises soy molasses.

2. The method of claim 1 further comprising recovering the resulting lactic acid and/or lactate from the medium.

3. The method of claim 2 further comprising isolating and/or purifying lactic acid and/or lactate.

4. The method of claim 1 any of the previous claims, wherein the lactic acid is optically pure lactic acid isomer, optionally L-lactic acid isomer.

5. The method of claim 1 further comprising preparing polylactic acid from the obtained lactic acid.

6. The method of claim 1, wherein the carbon substrate comprises galacto-oligosaccharides at least about 10%, 20%, 30%, 40%, 50%, 60%, 70%, 80%, or 90% by weight of the total carbohydrates in said carbon substrate.

7. The method of claim 1, wherein the galacto-oligosaccharide is selected from the group consisting of melibiose, manninotriose, manninotetraose, raffinose, stachyose, verbascose, ajucose, galactinol, digalactosyl myo-inositol, galactopinitol A, galactopinitol B, ciceritol, fagopyritol B1, fagopyritol B2 and any combination thereof.

8. The method of claim 1, wherein the carbon substrate comprises glucose, fructose, galactose, sucrose, lactose, maltose, starch, cellulose and/or any combination thereof.

9. The method of claim 1, wherein the carbon substrate comprises carbon substrates from legumes, soya, fava bean, peas, chickpeas, corn, sugarcane, sugar beets, lignocellulose or any combination thereof;
the carbon substrate comprises sugarcane molasses, sugar beet molasses and/or citrus molasses;
and/or the medium or carbon substrate comprises 5-100 wt % soy molasses.

10. A genetically modified fungus for producing lactic acid and/or lactate from a carbon substrate comprising soy molasses, wherein the fungus has been genetically modified to overexpress a gene encoding a lactate dehydrogenase and a gene encoding an alfa-galactosidase.

11. The method of claim 1 or the genetically modified fungus of claim 10, wherein the alfa-galactosidase enzyme is a heterologous alfa-galactosidase enzyme.

12. The method of claim 1 or the genetically modified fungus of claim 10, wherein the heterologous alfa-galactosidase enzyme is an alfa-galactosidase enzyme of a yeast or filamentous fungus, e.g. selected from the genera Aspergillus, Gibberella, Cunninghamella, Fusarium, Glomus, Humicola, Mortierella, Mucor, Penicillium, Pythium, Rhizomucor, Rhizopus, Trichoderma and Saccharomyces, specifically from the group consisting of Gibberella zeae, Gibberella intermedia, Gibberella moniliformis, Gibberella fujikuroi, Gibberella nygamai, Gibberella sp. F75, Fusarium sp. 2 F75, Fusarium oxysporum, Fusarium mangiferae, Fusarium proliferatum, Fusarium verticilloides, Aspergillus nidulans, Aspergillus oryzae, Aspergillus terreus, Aspergillus niger, Aspergillus fischeri, Rhizopus miehei, Rhizomucor miehei, Rhizopus oryzae, Trichoderma reesei, Trichoderma harzianum, Trichoderma longibrachiatum and Saccharomyces cerevisiae.

13. The method of claim 1 or the genetically modified fungus of claim 10, wherein the lactate dehydrogenase enzyme is a heterologous lactate dehydrogenase enzyme.

14. The method of claim 1 or the genetically modified fungus of claim 10, wherein the lactate dehydrogenase enzyme is heterologous lactate dehydrogenase enzyme from an organism, micro-organism, fungus, unicellular protozoan parasite, or bacterium, optionally from Bos, Kluyveromyces, Rhizopus, Plasmodium, Lactobacillus, Pediococcus or Bacillus.

15. The method of claim 1 or the genetically modified fungus of claim 10, wherein said modified fungus comprises one or more copies of a gene encoding a lactate dehydrogenase and/or a gene encoding an alfa-galactosidase.

16. The method of claim 1 or the genetically modified fungus of claim 10, wherein the gene encoding a lactate dehydrogenase is selected from the group consisting of ldh1, ldh2, ldh3, ldh4, ldh5, ldh6A, ldh6B, ldhA, ldhB, ldhC and ldhL, and/or
the gene encoding an alfa-galactosidase is selected from the group consisting of agl1, agl2, agl3, aglA, aglB, aglC, aglD, MEL1, MEL2, MEL5, and MEL6.

17. The method of claim 1 or the genetically modified fungus of claim 10, wherein the fungus has further been genetically modified to decrease ethanol production.

18. The method of claim 1 or the genetically modified fungus of claim 10, wherein the fungus has further been genetically modified to decrease ethanol production by modifying or deleting at least part of a gene associated with ethanol production or by inactivating a gene associated with ethanol production, and optionally the gene associated with ethanol production is selected from the group consisting of PDC1, PDC5, PDC6, ADH1, ADH2, ADH3, ADH4, ADH5, and any combination thereof.

19. The method of claim 1 or the genetically modified fungus of claim 10, wherein the fungus further comprises a genetic modification of one or more genes selected from the group consisting of CYB2, GPD1, GPD2, GPP1, GPP2, and any combination thereof.

20. The method of claim 1 or the genetically modified fungus of claim 10, wherein the fungus is a yeast or filamentous fungus.

21. The method claim 1 or the genetically modified fungus of claim 10, wherein the fungus is a yeast or filamentous fungus selected from the genera Aspergillus, Saccharomyces, Kluyveromyces, Pichia, Hansenula, Candida, Trichosporon, Rhizopus, Torulaspora, Issatchenkia and Scheffersomyces, e.g. specifically from the group consisting of Saccharomyces cerevisiae, S. uvarum, Kluyveromyces thermotolerans, K. lactis, K. marxianus, Hansenula polymorpha, Scheffersomyces stipitis, Rhizopus oryzae, Torulaspora pretoriensis, Issatchenkia orientalis, Pichia fermentans, P. galeiformis, P. deserticola, P. membranifaciens, P. jadinii, P. kudriavzevii, P. anomala, Candida ethanolica, C. sonorensis and C. apicola.

22. The method of claim 1 or the genetically modified fungus of claim 10 any, wherein the fungus has been deposited to the VTT Collection under the accession number VTT C-191026 or the accession number VTT C-201040.

23. A method of preparing the genetically modified fungus of claim 10, wherein said method comprises providing a fungus and genetically modifying the fungus to overexpress a gene encoding a lactate dehydrogenase and a gene encoding an alfa-galactosidase.

24. (canceled)

25. A method of producing one or more products selected from the group consisting of polymers, polyesters and polylactic acids, said method comprising culturing the genetically modified fungus of claim 10 in a galacto-oligosaccharides containing medium to produce lactic acid from a carbon substrate comprising soy molasses, recovering the resulting lactic acid and utilizing the recovered lactic acid in production of polymers, polyesters and/or polylactic acids.


SEQUENCE LISTING (38 sequences)

SEQ ID NO: 1 - protein, 747 aa, Aspergillus niger
MIGSSHAVVALGLFTLYGHSAAAPAIGASNSQTIVTNGTSFALNGDNVSYRFHVNSSTGDLISDHFGGVVSGTIPSPVEPAVNGWVGMPGRIRREFPDQGRGDFRIPAVRIRESAGYTVSDLQYVSHEVIEGKYALPGLPATFGDAQDATTLVVHLYDNYSSVAADLSYSIFPKYDAIVRSVNVTNQGPGNITIEALASISIDFPYEDLDMVSLRGDWAREANVQRSKVQYGVQGFGSSTGYSSHLHNPFLAIVDPATTESQGEAWGFNLVYTGSFSAQVEKGSQGFTRALLGFNPDQLSWNLGPGETLTSPECVAVYSDKGLGSVSRKFHRLYRNHLMKSKFATSDRPVLLNSWEGVYFDYNQSSIETLAEESAALGVHLFVMDDGWFGDKYPRVSDNAGLGDWMPNPARFPDGLTPVVQDITNLTVNGTESTKLRFGIWVEPEMVNPNSTLYHEHPEWALHAGPYPRTERRNQLVLNLALPAVQDFIIDFMTNLLQDTGISYVKWDNNRGIHETPSPSTDHQYMLGLYRVFDTLTTRFPDVLWEGCASGGGRFDAGMLQYVPQIWTSDNTDAIDRITIQFGTSLAYPPSAMGAHLSAVPNAQTGRTVPFTFRAHVAMMGGSFGLELDPATVEGDEIVPELLALAEKVNPIILNGDLYRLRLPQDSQWPAALFVSQDGAQAVLFYFQVQPNVNHAVPWVRLQGLDPKADYTVDGDQTYSGATLMNLGLQYSFDTEYGSKVVFLERQ

SEQ ID NO: 2 - protein, 444 aa, Trichoderma reesei
MTPHSIDRAARPSVWSGLALLLSTAHAIVMPDGVTGKVPSLGWNSWNAYHCDIDESKFLSAAEVIVSSGLLDAGYNYVNIDDCWSMKDGRVDGHIAVNTTRFPDGIDGLAKKVHDLGLKLGIYSTAGTATCAGYPASLGYEDVDAADFADWGVDYLKYDNCNVPSDWQDEYVACAPDAVQTGPNGTCSTALEPNLAPPGYDWSTSKSAERFNAMRNALAKQSREIVLSLCIWGVADVFSWGNETGISWRMSGDISPEWGSVTHIINMNSFKMNSVGFWGHNDADILEVGNGNLTAAETRTHFALWAAMKSPLLIGTDLAQLSQENIELLKNKHLLAFNQDSVYGQPATPYKWGVNPDWTFNYTNPAEYWAGPSSKGHLVLMMNTLDHTVRKEAKWSEIPGLSAGRYEVRDVWTDKSLGCLSSYKTAVAAHDTAVILVGKKCRNW

SEQ ID NO: 3 - protein, 751 aa, Rhizomucor miehei
MLRCVLSGLATSTLVCSLLVSAALDTGIHKHPDLDTWFLVTDKSTYVIGATAEGYVCNVYWGSRLEHMDNLNATLPAYTSSQNPPITYATEELPAFGGLRYRENLLDVELPDGVRELNLLYNGKTNTTGDNHLDVELVDGNRTDLTVTLHYELDIENDIIRRSYTIRNGLKKRVNLSRAQSAAWHPPTALGVDDTRELLTVAGEWGNEAQTQRTILRPGLTHVIQSKRGHTAHQSYPFFALRQVPSDLSPASNEVYFGALAWSGSWEITVDMNIYGYSRITGGIHHHDFGWTLEPGESFTVPEFAAGYTQDGIPGARKLLPRHVRKYQLKNVKTQQKKDMYNPVLYNSWEALTFNITYDKQIALADKAAPMGIELFAVDDGWFGARDNDSAGLGDWFVSKKKFPEGMKPLADHVHKLGMKFGLWFEPEAVNPDSDLYREHPDWVFFYDGIPRYTGRNQLQLNLGLKEVREYLYDRLSTLIREVGIDYIKWDMNRPFAEVTMHHYKRNPREAWVHIVTGFYSIVDRLKKEFPELLIETCSSGGGRMDIGILQKVDNAWTSDNTRPDARLFIQHGASLFLPPRIMYGWVTDSPSDSQIDIPLSFRFHVSYMGGLGIGSNLNTMTEQEIKEATGWIKLYKQIRHIMQNGDLDWLVNPSRVGDLIAATQTTTQDQSEAVVLAFRYNSVFSNALNPIRLRYLEPSAIYRVLVYEEDPEIVAEDYDMSGALLMNKGISLDGLNNIMFRSAVVWVQKK

SEQ ID NO: 4 - protein, 744 aa, Gibberella sp. F75
MVLVTLRGITTTAVLFCQAISALAESSDPIHVDGTSFALNGDNVSYRFHVDNTTGDLINDHYGGPVAEDGITAEIGPIQGWVNLIGRVRREFPDHGRGDFRIPAFQLQQASGTTVTDFRYKSHEVVEGKPGLPGLPSTFGEADDVSTLVVRMYDNYSSIAVDLSYSIFPKYDAVVRSVNITNRGNATVNLKRVSSWSVDLQQDNLDLIEIRGDWAREGMRVRRKVDFGTQGFQSSTGYSSHLHNPFLALVASTTTETQGEAWGFSLVYTGSFAVDVEKSSQGLTRAILGVNSLDFSWPLKPGQTFTTPEVVSVFSNKGVGGMSRQFHRLYRKHLMKSKYAEETRPVLLNSWEGLGFEINETAIEKIAKQSADLGIKLFVMDDGWFGNKYPRVNDSAGLGDWQPNKERFPDGLTPLVENITELRIANASDDLKFGIWFEPEMVNPKSDLYDKHPDWAIHAGSYPRTETRNQLVLNVALPEVQEFIIDSVSKILRESPISYVKWDNNRGIHETPDPTLNYKYMLGLYHVFETLTSRFPDVLWEGCASGGGRFDPGVLQWFPQIWTSDDTDAVERIAIQFGTSLAYPPSAMGAHLSHVPNGNTQRITSVKFRAHVAMMGGSFGVELDPSDLEPEEREQIPGLIELSEKINPIVITGDFYRLALPEETNYPAGQFISEDGKKVVLFAFQTRATINNSWPWFRLQGLDASAKYRVDNNQTVSGSTLMNMGIQLTFEGDYDSHVLMIEKQ

SEQ ID NO: 5 - protein, 481 aa, Aspergillus fischeri
MGLSSLLLSAALFGLKASALNNGLARTPQMGWNTWNSFACELNETVILNAAERIVSLGFRDLGYEYVVLDDCWSAGRNSSGYLIADSEKFPNGIAHLADKVHELGLKIGIYSSAGTWTCARYEGSLGYEEKDAALWASWGIDYLKYDNCYNEGEEGTPKLSFDRYNAMFKALNATGRPMLYSLCNWGVDGPWNFAPTIANSWRTAGDLSNVWDRDDVNCPCSELEGLDCKTPGYKCSIMNVLNKAVYYPSKAIPGAWNDLDMLQVGNGGLTDDESIAHMSLWAALKSPLLMTNVMTKIDPPTLSILQNPAVLAVSQDPVASTPVRQWRYFVDDVDENGKGEIQMYSGPLSGGDQLVLLLNAGSKAREMNATLVDIFWESGPKGTAKQVKQHWDVYDLWANRMSNEDAAAIINGTFTGPSPYNLTAMGGAHEVYSRPLPSNSKVLMGSKVGSVQPSGTVTAHVRPHGIAMLRLRATDKKDEL

SEQ ID NO: 6 - protein, 471 aa, Saccharomyces cerevisiae
MFAFYFLTACTTLKGVFGVSPSYNGLGLTPQMGWDSWNTFACDVSEQLLLDTADRISDLGLKDMGYKYVILDDCWSSGRDSDGFLVADKHKFPNGMGHVADHLHNNSFLFGMYSSAGEYTCAGYPGSLGREEEDAQFFANNRVDYLKYDNCYNKGQFGTPDVSYHRYKAMSDALNKTGRPIFYSLCNWGQDLTFYWGSGIANSWRMSGDITAEFTRPDSRCPCDGDEYDCKYAGFHCSIMNILNKAAPMGQNAGVGGWNDLDNLEVGVGNLTDDEEKAHFSMWAMVKSPLIIGADVNHLKASSYSIYSQASVIAINQDPKGIPATRVWRYYVSDTDEYGQGEIQMWSGPLDNGDQVVALLNGGSVARPMNTTLEEIFFDSNLGSKELTSTWDIYDLWANRVDNSTASAILEQNKAATGILYNATEQSYKDGLSKNDTRLFGQKIGSLSPNAILNTTVPAHGIAFYRLRPSA

SEQ ID NO: 7 - DNA, 2244 bp, Aspergillus niger
atgattggttcttcacatgcagttgttgctttgggtttgtttactttatatggtcattcagctgcagctccagcaattggtgcttctaattcacaaactatcgttacaaacggtacttcattcgctttgaacggtgacaacgtttcttacagattccatgttaattcttcaactggtgacttgatttctgatcattttggtggtgttgtttctggtactattccatcaccagttgaaccagctgttaatggttgggttggtatgccaggtagaattagaagagaatttccagatcaaggtagaggtgactttagaataccagcagttagaattagagaatctgctggttatacagtttcagatttgcaatacgtttctcatgaagttattgagggtaaatacgcattgccaggtttaccagctacttttggtgacgcacaagatgctactacattggttgttcatttgtacgataactactcttcagttgcagctgatttgtcttactcaatcttcccaaagtacgatgctatcgttagatcagttaacgttacaaaccaaggtccaggcaacatcactatcgaagcattggcttctatctcaatcgatttcccatacgaagatttggatatggtttcattaagaggtgactgggcaagagaagctaacgttcaaagatcaaaggttcaatatggtgttcaaggttttggttcttcaactggttactcttcacatttgcataacccatttttagcaatcgttgatccagctactacagaatctcaaggtgaagcttggggttttaatttggtttacacaggttctttttcagcacaagttgaaaaaggttcacaaggttttactagagctttgttaggttttaatccagatcaattgtcttggaatttgggtccaggtgaaactttaacatcaccagaatgtgttgctgtttattctgataaaggtttgggttctgtttcaagaaagttccatagattgtacagaaaccatttgatgaagtctaagttcgctacatcagatagaccagttttgttaaattcatgggaaggtgtttacttcgattacaaccaatcttcaatcgaaactttagcagaagaatctgcagctttgggtgttcatttgttcgttatggatgatggttggtttggtgacaaatatccaagagtttctgataatgctggtttgggtgactggatgccaaatccagctagatttccagatggtttgacaccagttgttcaagatatcacaaatttgactgttaacggtacagaatcaactaagttgagattcggtatttgggttgaaccagaaatggttaacccaaattctacattgtaccatgaacatccagaatgggcattacatgctggtccatacccaagaactgaaagaagaaaccaattggttttgaatttggcattaccagctgttcaagatttcatcatcgatttcatgacaaatttgttgcaagatactggtatctcttacgttaagtgggataacaacagaggtattcatgaaacaccatctccatcaactgatcatcaatacatgttgggtttatacagagttttcgatactttgactacaagattcccagatgttttatgggaaggttgtgcttctggtggtggtagatttgatgctggcatgttacaatatgttccacaaatttggacatctgataacactgatgcaatcgatagaatcacaattcaatttggtacttcattagcttacccaccatctgcaatgggtgctcatttgtcagcagttccaaatgctcaaacaggtagaactgttccttttacttttagagctcatgttgctatgatgggtggttcttttggtttggaattagatccagctactgttgaaggtgacgaaattgttccagaattgttagcattggctgaaaaggttaacccaatcatcttgaacggtgacttgtacagattgagattaccacaagattcacaatggccagcagctttatttgtttctcaagatggtgcacaagctgttttgttttacttccaagttcaaccaaacgttaaccatgcagttccatgggttagattgcaaggtttagatccaaaagctgattatacagttgatggtgaccaaacatactctggtgcaactttaatgaatttgggtttacaatactcattcgatactgaatacggttctaaagttgttttcttggaaagacaataa

SEQ ID NO: 8 - DNA, 1335 bp, Trichoderma reesei
atgacaccacattctattgatagagctgcaagaccatcagtttggtctggtttggctttgttattgtcaactgctcatgcaattgttatgccagatggtgttactggtaaagttccatctttgggttggaactcttggaatgcatatcattgtgatatcgatgaatcaaagttcttgtctgctgcagaagttattgtttcttcaggtttattggatgctggttacaactacgttaacatcgatgattgttggtctatgaaggatggtagagttgatggtcatattgcagttaatactacaagatttccagatggtattgatggtttggctaagaaagttcatgatttgggtttgaagttgggtatctattctacagctggtactgcaacatgtgctggttatccagcatcattgggttacgaagatgttgatgctgcagattttgctgattggggtgttgattatttgaagtacgataactgtaacgttccatctgattggcaagatgaatatgttgcttgtgcaccagatgctgttcaaactggtccaaatggtacttgttcaacagcattggaaccaaatttggctccaccaggttacgattggtctacatcaaaatctgcagaaagattcaatgctatgagaaatgctttggcaaagcaatcaagagaaatcgttttgtcattatgtatttggggtgttgctgatgttttctcttggggtaatgaaactggtatttcatggagaatgtctggtgacatttcaccagaatggggttcagttacacatattattaacatgaactcttttaaaatgaactctgttggtttctggggtcataatgatgctgatattttggaagttggtaacggtaatttgactgctgcagaaactagaacacattttgcattatgggctgcaatgaaatctccattattgattggtacagatttggctcaattatcacaagaaaacatcgaattgttgaaaaataagcatttgttggcttttaatcaagattctgtttatggtcaaccagctactccatacaaatggggtgttaacccagattggacttttaattacacaaacccagcagaatactgggctggtccatcttcaaagggtcatttggttttgatgatgaacactttggatcatacagttagaaaggaagcaaaatggtctgaaattccaggtttatcagctggtagatatgaagttagagatgtttggacagataaatctttgggttgtttatcttcatacaaaactgcagttgctgcacatgatacagctgttattttggttggtaaaaagtgtagaaactggtaa

SEQ ID NO: 9 - DNA, 2256 bp, Rhizomucor miehei
atgttaagatgtgttttgtctggtttagctacatcaactttagtttgttctttgttagtttcagctgcattggatacaggtattcataaacatccagatttggatacttggttcttggttactgataagtctacttacgttatcggtgctactgcagaaggttatgtttgtaatgtttactggggttcaagattggaacatatggataatttgaatgctacattaccagcatacacttcttcacaaaatccaccaattacatacgctactgaagaattaccagcattcggtggtttgagatatagagaaaatttgttagatgttgaattgccagatggtgttagagaattgaatttgttgtacaacggtaaaacaaacactacaggtgacaaccatttggatgttgaattagttgatggtaacagaactgatttgacagttactttacattacgaattggatatcgaaaacgatatcattagaagatcatacacaatcagaaacggtttgaagaaaagagttaatttgtcaagagctcaatcagctgcatggcatccaccaactgcattaggtgttgatgatacaagagaattgttaactgttgctggtgaatggggtaacgaagcacaaacacaaagaactatcttaagaccaggtttgacacatgttattcaatctaaaagaggtcatactgctcatcaatcatatccatttttcgcattaagacaagttccatcagatttgtctccagcttcaaatgaagtttactttggtgctttagcatggtctggttcatgggaaatcacagttgatatgaacatctatggttattcaagaatcactggtggtattcatcatcatgattttggttggacattggaacctggtgaatcttttactgttccagaatttgctgcaggttatacacaagatggtattccaggtgctagaaagttgttgccaagacatgttagaaagtaccaattgaaaaatgttaaaactcaacaaaagaaagatatgtacaacccagttttgtacaactcttgggaagctttgacttttaatatcacttacgataagcaaatcgctttagcagataaagctgcaccaatgggtattgaattgtttgcagttgatgatggttggtttggtgctagagataatgattctgctggtttgggtgactggttcgtttctaaaaagaaattcccagagggtatgaaaccattggctgatcatgttcataagttgggtatgaagttcggtttgtggtttgaaccagaagcagttaatccagattctgatttgtatagagaacatccagattgggttttcttttatgatggtattccaagatacacaggtagaaaccaattgcaattgaatttgggtttaaaagaagttagagaatatttgtacgatagattgtcaactttaatcagagaagttggtatcgattacatcaagtgggatatgaacagaccattcgctgaagttacaatgcatcattacaagagaaacccaagagaagcatgggttcatatcgttactggtttctactctatcgttgatagattgaagaaagaatttccagaattgttgattgaaacatgttcttctggtggtggtagaatggatatcggtattttacaaaaggttgataacgcttggacatctgataatactagaccagatgctagattgtttattcaacatggtgcatcattgtttttaccaccaagaatcatgtatggttgggttactgattctccatcagattctcaaatcgatatcccattgtcttttagattccatgtttcatacatgggtggtttaggtatcggttctaatttgaatacaatgactgaacaagaaattaaagaagctacaggttggatcaagttgtacaagcaaatcagacatatcatgcaaaacggtgacttggattggttggttaatccatcaagagttggtgacttgattgctgcaactcaaactacaactcaagatcaatcagaagctgttgttttggcttttagatacaactctgttttctctaacgctttgaacccaatcagattgagatacttagaaccatctgcaatctatagagttttagtttacgaagaagatccagaaattgttgctgaagattatgatatgtctggtgcattgttgatgaataagggtatctcattggatggtttaaataacatcatgtttagatcagctgttgtttgggttcaaaagaaataa

SEQ ID NO: 10 - DNA, 2235 bp, Gibberella sp. F75
atggttttggttactttaagaggtatcactacaactgctgttttattttgtcaagcaatctctgctttggcagaatcttcagatccaattcatgttgatggtacatctttcgctttgaacggtgacaacgtttcatacagattccatgttgataacacaactggtgacttgattaatgatcattacggtggtccagttgctgaagatggtattactgcagaaattggtccaattcaaggttgggttaatttgatcggtagagttagaagagaatttccagatcatggtagaggtgactttagaataccagcttttcaattgcaacaagcatctggtacaactgttacagatttcagatacaaatcacatgaagttgttgaaggtaaaccaggtttgccaggtttaccatctacttttggtgaagctgatgatgtttcaacattagttgttagaatgtacgataactactcttcaatcgctgttgatttgtcttactcaatcttcccaaagtacgatgcagttgttagatcagttaacatcactaaccgtggtaacgctacagttaatttgaagagagtttcttcatggtctgttgatttgcaacaagataatttggatttgatcgaaatcagaggtgactgggcaagagaaggcatgagagttagaagaaaagttgattttggtactcaaggttttcaatcttcaacaggttattcttctcatttgcataacccatttttggctttagttgcatctacaactacagaaactcaaggtgaagcttggggtttttctttggtttacacaggttcatttgcagttgatgttgaaaaatcttcacaaggtttgactagagctattttaggtgttaattctttggatttctcatggccattgaaaccaggtcaaacttttactactccagaagttgtttctgttttctctaataagggtgttggtggtatgtcaagacaattccatagattatacagaaagcatttgatgaagtctaagtacgcagaagaaactagaccagttttgttaaattcatgggaaggtttaggtttcgaaattaatgaaacagctatcgaaaagattgctaagcaatctgcagatttgggtattaaattgttcgttatggatgatggttggttcggtaataagtacccaagagttaacgattcagctggtttgggtgactggcaaccaaataaggaaagatttccagatggtttaactccattggttgaaaacatcacagaattgagaatcgctaacgcatctgatgatttgaagttcggtatctggttcgaaccagaaatggttaacccaaagtctgatttgtacgataagcatccagattgggctattcatgcaggttcatacccaagaactgaaacaagaaaccaattggttttgaatgttgctttgccagaagttcaagagtttattatcgattctgtttcaaaaattttgagagaatctccaatctcatacgttaagtgggataacaacagaggtattcatgaaactccagatccaacattaaactacaagtacatgttgggtttataccatgttttcgaaactttaacttcaagattcccagatgttttgtgggaaggttgtgcttctggtggtggtagatttgatccaggtgttttgcaatggtttccacaaatttggacttctgatgatacagatgctgttgaaagaatcgcaatccaattcggtacttctttagcttacccaccatcagctatgggtgcacatttgtctcatgttccaaacggtaacactcaaagaatcacatcagtcaagtttagagctcatgttgcaatgatgggtggttcttttggtgttgaattagatccatcagatttggaaccagaagaaagagaacaaatcccaggtttgatcgaattgtctgaaaagattaatccaatcgttatcactggtgacttctacagattggctttaccagaagaaacaaattacccagcaggtcaattcatttcagaagatggtaaaaaggttgttttgttcgctttccaaactagagcaacaattaataactcttggccatggttcagattgcaaggtttagatgcttcagcaaagtacagagttgataacaaccaaactgtttctggttcaacattaatgaacatgggtattcaattgacttttgagggtgactacgattctcatgttttgatgatcgaaaagcaataa

SEQ ID NO: 11 - DNA, 1446 bp, Aspergillus fischeri
atgggtttgtcttcattgttattgtcagctgcattgttcggtttaaaagcatctgctttgaacaacggtttagcaagaactccacaaatgggttggaatacatggaactcttttgcttgtgaattaaacgaaactgttattttgaacgctgcagaaagaatcgtttctttgggttttagagatttgggttacgaatacgttgttttagatgattgttggtcagcaggtagaaattcttcaggttatttgatcgctgattctgaaaagttcccaaacggtatcgcacatttggctgataaagttcatgaattgggtttaaagatcggtatatattcttctgcaggtacttggacatgtgctagatatgaaggttcattgggttacgaagaaaaagatgctgcattatgggcatcttggggtatcgactatttgaagtacgataactgttacaacgaaggtgaagaaggtactccaaagttgtcattcgatagatacaacgctatgttcaaagcattgaatgctacaggtagaccaatgttgtactctttgtgtaactggggtgttgatggtccatggaattttgcaccaactattgctaattcatggagaacagctggtgacttgtctaatgtttgggatagagatgatgttaattgtccatgttctgaattggaaggtttagattgtaaaactccaggttacaagtgttcaatcatgaacgttttgaataaggcagtttattacccatctaaagcaattccaggtgcttggaacgatttggatatgttgcaagttggtaacggtggtttaacagatgatgaatcaatcgctcatatgtctttgtgggctgcattgaaatcaccattattgatgactaacgttatgacaaagatcgatccaccaactttgtctatcttgcaaaacccagcagttttagctgtttcacaagatccagttgcttctacaccagttagacaatggagatacttcgttgatgatgttgatgaaaacggtaaaggtgaaatccaaatgtactctggtccattgtctggtggtgaccaattggttttgttgttgaacgcaggttcaaaggctagagaaatgaacgctactttggttgatattttctgggaatctggtccaaaaggtacagcaaagcaagttaagcaacattgggatgtttacgatttgtgggctaatagaatgtctaatgaagatgctgcagctattattaacggtacttttacaggtccatcaccatataatttgactgcaatgggtggtgctcatgaagtttattcaagaccattgccatctaattcaaaggttttgatgggttctaaagttggttcagttcaaccatctggtactgttacagctcatgttagaccacatggtattgcaatgttgagattaagagctacagataagaaagatgaattataa

SEQ ID NO: 12 - DNA, 1416 bp, Saccharomyces cerevisiae
atgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaa

SEQ ID NO: 13 - DNA, 48 bp, Artificial Sequence
  oligonucleotide 32 MEL5-ATG-F
acacaaacactaaatcaaagaattcatgtttgctttctactttctcac

SEQ ID NO: 14 - DNA, 47 bp, Artificial Sequence
  oligonucleotide 33 MEL5-stopR
aggttgagaccctcggcttaaggcgcgccctcgagagcttttgatta

SEQ ID NO: 15 - DNA, 8575 bp, Artificial Sequence
  pMIE-016_AnAglC, ligation of NoName*EcoRI+AscI 2251 bp into pMIE-005*EcoRI+AscI 6324 bp
caggatcccaattaatgtgagttacctcactcattaggcaccccaggctttacactttatgcttccggctcgtatgttgtgtggaattgtgagcggataacaatttcacacaggaaacagctatgaccatgattacgaattaattcgagctcggtacccggggatcgatccactagtcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaagaattcatgattggttcttcacatgcagttgttgctttgggtttgtttactttatatggtcattcagctgcagctccagcaattggtgcttctaattcacaaactatcgttacaaacggtacttcattcgctttgaacggtgacaacgtttcttacagattccatgttaattcttcaactggtgacttgatttctgatcattttggtggtgttgtttctggtactattccatcaccagttgaaccagctgttaatggttgggttggtatgccaggtagaattagaagagaatttccagatcaaggtagaggtgactttagaataccagcagttagaattagagaatctgctggttatacagtttcagatttgcaatacgtttctcatgaagttattgagggtaaatacgcattgccaggtttaccagctacttttggtgacgcacaagatgctactacattggttgttcatttgtacgataactactcttcagttgcagctgatttgtcttactcaatcttcccaaagtacgatgctatcgttagatcagttaacgttacaaaccaaggtccaggcaacatcactatcgaagcattggcttctatctcaatcgatttcccatacgaagatttggatatggtttcattaagaggtgactgggcaagagaagctaacgttcaaagatcaaaggttcaatatggtgttcaaggttttggttcttcaactggttactcttcacatttgcataacccatttttagcaatcgttgatccagctactacagaatctcaaggtgaagcttggggttttaatttggtttacacaggttctttttcagcacaagttgaaaaaggttcacaaggttttactagagctttgttaggttttaatccagatcaattgtcttggaatttgggtccaggtgaaactttaacatcaccagaatgtgttgctgtttattctgataaaggtttgggttctgtttcaagaaagttccatagattgtacagaaaccatttgatgaagtctaagttcgctacatcagatagaccagttttgttaaattcatgggaaggtgtttacttcgattacaaccaatcttcaatcgaaactttagcagaagaatctgcagctttgggtgttcatttgttcgttatggatgatggttggtttggtgacaaatatccaagagtttctgataatgctggtttgggtgactggatgccaaatccagctagatttccagatggtttgacaccagttgttcaagatatcacaaatttgactgttaacggtacagaatcaactaagttgagattcggtatttgggttgaaccagaaatggttaacccaaattctacattgtaccatgaacatccagaatgggcattacatgctggtccatacccaagaactgaaagaagaaaccaattggttttgaatttggcattaccagctgttcaagatttcatcatcgatttcatgacaaatttgttgcaagatactggtatctcttacgttaagtgggataacaacagaggtattcatgaaacaccatctccatcaactgatcatcaatacatgttgggtttatacagagttttcgatactttgactacaagattcccagatgttttatgggaaggttgtgcttctggtggtggtagatttgatgctggcatgttacaatatgttccacaaatttggacatctgataacactgatgcaatcgatagaatcacaattcaatttggtacttcattagcttacccaccatctgcaatgggtgctcatttgtcagcagttccaaatgctcaaacaggtagaactgttccttttacttttagagctcatgttgctatgatgggtggttcttttggtttggaattagatccagctactgttgaaggtgacgaaattgttccagaattgttagcattggctgaaaaggttaacccaatcatcttgaacggtgacttgtacagattgagattaccacaagattcacaatggccagcagctttatttgtttctcaagatggtgcacaagctgttttgttttacttccaagttcaaccaaacgttaaccatgcagttccatgggttagattgcaaggtttagatccaaaagctgattatacagttgatggtgaccaaacatactctggtgcaactttaatgaatttgggtttacaatactcattcgatactgaatacggttctaaagttgttttcttggaaagacaataaggcgcgccctcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcctaatgtgtcaatgatcatattcttaactggaccgatcttattcgtcagattcaaaccaaaagttcttagggctaccacaggaggaaaattagtgtgatataatttaaataatttatccgccattcctaatagaacgttgttcgacggatatctttctgcccaaaagggttctaagctcaatgaagagccaatgtctaaacctctttgcggccgcaagctaattcgcgcgaagctagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcataggagatctaagctctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaaaagctcttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatc

SEQ ID NO: 16 - DNA, 7666 bp, Artificial Sequence
  pMIE-017_TrAgl1, ligation of NoName* into pMIE-005*
caggatcccaattaatgtgagttacctcactcattaggcaccccaggctttacactttatgcttccggctcgtatgttgtgtggaattgtgagcggataacaatttcacacaggaaacagctatgaccatgattacgaattaattcgagctcggtacccggggatcgatccactagtcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaagaattcatgacaccacattctattgatagagctgcaagaccatcagtttggtctggtttggctttgttattgtcaactgctcatgcaattgttatgccagatggtgttactggtaaagttccatctttgggttggaactcttggaatgcatatcattgtgatatcgatgaatcaaagttcttgtctgctgcagaagttattgtttcttcaggtttattggatgctggttacaactacgttaacatcgatgattgttggtctatgaaggatggtagagttgatggtcatattgcagttaatactacaagatttccagatggtattgatggtttggctaagaaagttcatgatttgggtttgaagttgggtatctattctacagctggtactgcaacatgtgctggttatccagcatcattgggttacgaagatgttgatgctgcagattttgctgattggggtgttgattatttgaagtacgataactgtaacgttccatctgattggcaagatgaatatgttgcttgtgcaccagatgctgttcaaactggtccaaatggtacttgttcaacagcattggaaccaaatttggctccaccaggttacgattggtctacatcaaaatctgcagaaagattcaatgctatgagaaatgctttggcaaagcaatcaagagaaatcgttttgtcattatgtatttggggtgttgctgatgttttctcttggggtaatgaaactggtatttcatggagaatgtctggtgacatttcaccagaatggggttcagttacacatattattaacatgaactcttttaaaatgaactctgttggtttctggggtcataatgatgctgatattttggaagttggtaacggtaatttgactgctgcagaaactagaacacattttgcattatgggctgcaatgaaatctccattattgattggtacagatttggctcaattatcacaagaaaacatcgaattgttgaaaaataagcatttgttggcttttaatcaagattctgtttatggtcaaccagctactccatacaaatggggtgttaacccagattggacttttaattacacaaacccagcagaatactgggctggtccatcttcaaagggtcatttggttttgatgatgaacactttggatcatacagttagaaaggaagcaaaatggtctgaaattccaggtttatcagctggtagatatgaagttagagatgtttggacagataaatctttgggttgtttatcttcatacaaaactgcagttgctgcacatgatacagctgttattttggttggtaaaaagtgtagaaactggtaaggcgcgccctcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcctaatgtgtcaatgatcatattcttaactggaccgatcttattcgtcagattcaaaccaaaagttcttagggctaccacaggaggaaaattagtgtgatataatttaaataatttatccgccattcctaatagaacgttgttcgacggatatctttctgcccaaaagggttctaagctcaatgaagagccaatgtctaaacctctttgcggccgcaagctaattcgcgcgaagctagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcataggagatctaagctctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaaaagctcttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatc

SEQ ID NO: 17 - DNA, 8587 bp, Artificial Sequence
  pMIE-018_RmGal36, Ligation of NoName* into pMIE-005*
caggatcccaattaatgtgagttacctcactcattaggcaccccaggctttacactttatgcttccggctcgtatgttgtgtggaattgtgagcggataacaatttcacacaggaaacagctatgaccatgattacgaattaattcgagctcggtacccggggatcgatccactagtcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaagaattcatgttaagatgtgttttgtctggtttagctacatcaactttagtttgttctttgttagtttcagctgcattggatacaggtattcataaacatccagatttggatacttggttcttggttactgataagtctacttacgttatcggtgctactgcagaaggttatgtttgtaatgtttactggggttcaagattggaacatatggataatttgaatgctacattaccagcatacacttcttcacaaaatccaccaattacatacgctactgaagaattaccagcattcggtggtttgagatatagagaaaatttgttagatgttgaattgccagatggtgttagagaattgaatttgttgtacaacggtaaaacaaacactacaggtgacaaccatttggatgttgaattagttgatggtaacagaactgatttgacagttactttacattacgaattggatatcgaaaacgatatcattagaagatcatacacaatcagaaacggtttgaagaaaagagttaatttgtcaagagctcaatcagctgcatggcatccaccaactgcattaggtgttgatgatacaagagaattgttaactgttgctggtgaatggggtaacgaagcacaaacacaaagaactatcttaagaccaggtttgacacatgttattcaatctaaaagaggtcatactgctcatcaatcatatccatttttcgcattaagacaagttccatcagatttgtctccagcttcaaatgaagtttactttggtgctttagcatggtctggttcatgggaaatcacagttgatatgaacatctatggttattcaagaatcactggtggtattcatcatcatgattttggttggacattggaacctggtgaatcttttactgttccagaatttgctgcaggttatacacaagatggtattccaggtgctagaaagttgttgccaagacatgttagaaagtaccaattgaaaaatgttaaaactcaacaaaagaaagatatgtacaacccagttttgtacaactcttgggaagctttgacttttaatatcacttacgataagcaaatcgctttagcagataaagctgcaccaatgggtattgaattgtttgcagttgatgatggttggtttggtgctagagataatgattctgctggtttgggtgactggttcgtttctaaaaagaaattcccagagggtatgaaaccattggctgatcatgttcataagttgggtatgaagttcggtttgtggtttgaaccagaagcagttaatccagattctgatttgtatagagaacatccagattgggttttcttttatgatggtattccaagatacacaggtagaaaccaattgcaattgaatttgggtttaaaagaagttagagaatatttgtacgatagattgtcaactttaatcagagaagttggtatcgattacatcaagtgggatatgaacagaccattcgctgaagttacaatgcatcattacaagagaaacccaagagaagcatgggttcatatcgttactggtttctactctatcgttgatagattgaagaaagaatttccagaattgttgattgaaacatgttcttctggtggtggtagaatggatatcggtattttacaaaaggttgataacgcttggacatctgataatactagaccagatgctagattgtttattcaacatggtgcatcattgtttttaccaccaagaatcatgtatggttgggttactgattctccatcagattctcaaatcgatatcccattgtcttttagattccatgtttcatacatgggtggtttaggtatcggttctaatttgaatacaatgactgaacaagaaattaaagaagctacaggttggatcaagttgtacaagcaaatcagacatatcatgcaaaacggtgacttggattggttggttaatccatcaagagttggtgacttgattgctgcaactcaaactacaactcaagatcaatcagaagctgttgttttggcttttagatacaactctgttttctctaacgctttgaacccaatcagattgagatacttagaaccatctgcaatctatagagttttagtttacgaagaagatccagaaattgttgctgaagattatgatatgtctggtgcattgttgatgaataagggtatctcattggatggtttaaataacatcatgtttagatcagctgttgtttgggttcaaaagaaataaggcgcgccctcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcctaatgtgtcaatgatcatattcttaactggaccgatcttattcgtcagattcaaaccaaaagttcttagggctaccacaggaggaaaattagtgtgatataatttaaataatttatccgccattcctaatagaacgttgttcgacggatatctttctgcccaaaagggttctaagctcaatgaagagccaatgtctaaacctctttgcggccgcaagctaattcgcgcgaagctagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcataggagatctaagctctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaaaagctcttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatc

SEQ ID NO: 18 - DNA, 8566 bp, Artificial Sequence
  pMIE-019_GibGal3, ligation of NoName* into pMIE-005*
caggatcccaattaatgtgagttacctcactcattaggcaccccaggctttacactttatgcttccggctcgtatgttgtgtggaattgtgagcggataacaatttcacacaggaaacagctatgaccatgattacgaattaattcgagctcggtacccggggatcgatccactagtcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaagaattcatggttttggttactttaagaggtatcactacaactgctgttttattttgtcaagcaatctctgctttggcagaatcttcagatccaattcatgttgatggtacatctttcgctttgaacggtgacaacgtttcatacagattccatgttgataacacaactggtgacttgattaatgatcattacggtggtccagttgctgaagatggtattactgcagaaattggtccaattcaaggttgggttaatttgatcggtagagttagaagagaatttccagatcatggtagaggtgactttagaataccagcttttcaattgcaacaagcatctggtacaactgttacagatttcagatacaaatcacatgaagttgttgaaggtaaaccaggtttgccaggtttaccatctacttttggtgaagctgatgatgtttcaacattagttgttagaatgtacgataactactcttcaatcgctgttgatttgtcttactcaatcttcccaaagtacgatgcagttgttagatcagttaacatcactaaccgtggtaacgctacagttaatttgaagagagtttcttcatggtctgttgatttgcaacaagataatttggatttgatcgaaatcagaggtgactgggcaagagaaggcatgagagttagaagaaaagttgattttggtactcaaggttttcaatcttcaacaggttattcttctcatttgcataacccatttttggctttagttgcatctacaactacagaaactcaaggtgaagcttggggtttttctttggtttacacaggttcatttgcagttgatgttgaaaaatcttcacaaggtttgactagagctattttaggtgttaattctttggatttctcatggccattgaaaccaggtcaaacttttactactccagaagttgtttctgttttctctaataagggtgttggtggtatgtcaagacaattccatagattatacagaaagcatttgatgaagtctaagtacgcagaagaaactagaccagttttgttaaattcatgggaaggtttaggtttcgaaattaatgaaacagctatcgaaaagattgctaagcaatctgcagatttgggtattaaattgttcgttatggatgatggttggttcggtaataagtacccaagagttaacgattcagctggtttgggtgactggcaaccaaataaggaaagatttccagatggtttaactccattggttgaaaacatcacagaattgagaatcgctaacgcatctgatgatttgaagttcggtatctggttcgaaccagaaatggttaacccaaagtctgatttgtacgataagcatccagattgggctattcatgcaggttcatacccaagaactgaaacaagaaaccaattggttttgaatgttgctttgccagaagttcaagagtttattatcgattctgtttcaaaaattttgagagaatctccaatctcatacgttaagtgggataacaacagaggtattcatgaaactccagatccaacattaaactacaagtacatgttgggtttataccatgttttcgaaactttaacttcaagattcccagatgttttgtgggaaggttgtgcttctggtggtggtagatttgatccaggtgttttgcaatggtttccacaaatttggacttctgatgatacagatgctgttgaaagaatcgcaatccaattcggtacttctttagcttacccaccatcagctatgggtgcacatttgtctcatgttccaaacggtaacactcaaagaatcacatcagtcaagtttagagctcatgttgcaatgatgggtggttcttttggtgttgaattagatccatcagatttggaaccagaagaaagagaacaaatcccaggtttgatcgaattgtctgaaaagattaatccaatcgttatcactggtgacttctacagattggctttaccagaagaaacaaattacccagcaggtcaattcatttcagaagatggtaaaaaggttgttttgttcgctttccaaactagagcaacaattaataactcttggccatggttcagattgcaaggtttagatgcttcagcaaagtacagagttgataacaaccaaactgtttctggttcaacattaatgaacatgggtattcaattgacttttgagggtgactacgattctcatgttttgatgatcgaaaagcaataaggcgcgccctcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcctaatgtgtcaatgatcatattcttaactggaccgatcttattcgtcagattcaaaccaaaagttcttagggctaccacaggaggaaaattagtgtgatataatttaaataatttatccgccattcctaatagaacgttgttcgacggatatctttctgcccaaaagggttctaagctcaatgaagagccaatgtctaaacctctttgcggccgcaagctaattcgcgcgaagctagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcataggagatctaagctctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaaaagctcttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatc

SEQ ID NO: 19 - DNA, 7777 bp, Artificial Sequence
  pMIE-020_AfGal27, ligation of NoName* into pMIE-005*
caggatcccaattaatgtgagttacctcactcattaggcaccccaggctttacactttatgcttccggctcgtatgttgtgtggaattgtgagcggataacaatttcacacaggaaacagctatgaccatgattacgaattaattcgagctcggtacccggggatcgatccactagtcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaagaattcatgggtttgtcttcattgttattgtcagctgcattgttcggtttaaaagcatctgctttgaacaacggtttagcaagaactccacaaatgggttggaatacatggaactcttttgcttgtgaattaaacgaaactgttattttgaacgctgcagaaagaatcgtttctttgggttttagagatttgggttacgaatacgttgttttagatgattgttggtcagcaggtagaaattcttcaggttatttgatcgctgattctgaaaagttcccaaacggtatcgcacatttggctgataaagttcatgaattgggtttaaagatcggtatatattcttctgcaggtacttggacatgtgctagatatgaaggttcattgggttacgaagaaaaagatgctgcattatgggcatcttggggtatcgactatttgaagtacgataactgttacaacgaaggtgaagaaggtactccaaagttgtcattcgatagatacaacgctatgttcaaagcattgaatgctacaggtagaccaatgttgtactctttgtgtaactggggtgttgatggtccatggaattttgcaccaactattgctaattcatggagaacagctggtgacttgtctaatgtttgggatagagatgatgttaattgtccatgttctgaattggaaggtttagattgtaaaactccaggttacaagtgttcaatcatgaacgttttgaataaggcagtttattacccatctaaagcaattccaggtgcttggaacgatttggatatgttgcaagttggtaacggtggtttaacagatgatgaatcaatcgctcatatgtctttgtgggctgcattgaaatcaccattattgatgactaacgttatgacaaagatcgatccaccaactttgtctatcttgcaaaacccagcagttttagctgtttcacaagatccagttgcttctacaccagttagacaatggagatacttcgttgatgatgttgatgaaaacggtaaaggtgaaatccaaatgtactctggtccattgtctggtggtgaccaattggttttgttgttgaacgcaggttcaaaggctagagaaatgaacgctactttggttgatattttctgggaatctggtccaaaaggtacagcaaagcaagttaagcaacattgggatgtttacgatttgtgggctaatagaatgtctaatgaagatgctgcagctattattaacggtacttttacaggtccatcaccatataatttgactgcaatgggtggtgctcatgaagtttattcaagaccattgccatctaattcaaaggttttgatgggttctaaagttggttcagttcaaccatctggtactgttacagctcatgttagaccacatggtattgcaatgttgagattaagagctacagataagaaagatgaattataaggcgcgccctcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcctaatgtgtcaatgatcatattcttaactggaccgatcttattcgtcagattcaaaccaaaagttcttagggctaccacaggaggaaaattagtgtgatataatttaaataatttatccgccattcctaatagaacgttgttcgacggatatctttctgcccaaaagggttctaagctcaatgaagagccaatgtctaaacctctttgcggccgcaagctaattcgcgcgaagctagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcataggagatctaagctctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaaaagctcttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatc

SEQ ID NO: 20 - DNA, 7747 bp, Artificial Sequence
  pMIE-005, ligation of i-pMLV18 (B3661)** into pMI529**
caggatcccaattaatgtgagttacctcactcattaggcaccccaggctttacactttatgcttccggctcgtatgttgtgtggaattgtgagcggataacaatttcacacaggaaacagctatgaccatgattacgaattaattcgagctcggtacccggggatcgatccactagtcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaagaattcatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaaggcgcgccctcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcctaatgtgtcaatgatcatattcttaactggaccgatcttattcgtcagattcaaaccaaaagttcttagggctaccacaggaggaaaattagtgtgatataatttaaataatttatccgccattcctaatagaacgttgttcgacggatatctttctgcccaaaagggttctaagctcaatgaagagccaatgtctaaacctctttgcggccgcaagctaattcgcgcgaagctagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcctgatgcggtattttctccttacgcatctgtgcggtatttcacaccgcataggagatctaagctctggcgtaatagcgaagaggcccgcaccgatcgcccttcccaacagttgcgcagcctgaatggcgaatggcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaaaagctcttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatc

SEQ ID NO: 21 - DNA, 90 bp, Artificial Sequence
  oligonucleotide 2ScADH1 -150F
gtttgctgtcttgctatcaagtataaatagacctgcaattattaatcttttgtttcctcgtcgcacttcgtacgctgcaggtcgacaacc

SEQ ID NO: 22 - DNA, 88 bp, Artificial Sequence
  oligonucleotide 5ScADH1 stopR
atcataaatcataagaaattcgcttatttagaagtgtcaacaacgtatctaccaacgatttgacggccactagtggatctgatatcac

SEQ ID NO: 23 - DNA, 7488 bp, Artificial Sequence
  pMIE-021B
gtcttgtatcattcttagacaaaccgtctttataagactgctctgtagcattgtagagaataccggtggctgccttattctgttcaaggatagcagacgccgtagagttgtcaactctgttggcccataagtcgtaaatatcccaagtcgatgtcagttcctttgaacccaaattgctgtcaaagaaaatctcttccaaggtcgtgttcattggtcttgctacgcttcctccattcaataaagcaaccacttggtcaccattgtcaagcggaccactccacatttgaatttcaccttgtccatattcatcggtgtctgaaacataatatctccagactcttgtggctggaataccctttggatcttgattaattgcgatgacagaggcttgactgtagatcgagtacgaagatgcctttaagtgattcacgtcggcaccaatgataagtggggactttaccattgcccacatagagaaatgggccttttcctcatcgtcagtcaaattaccgactccgacctctagattgtccagatcgttccaaccaccaacacctgcattttgccccattggagctgccttgttaagaatattcataatagaacaatggaaaccggcgtacttgcaatcatattcgtcaccgtcacagggacatctgctatctggacgggtgaactcagcagtaatatctccgctcattctccaagaattggcgataccagagccccagtaaaatgtcaaatcctgaccccagttacatagagaatagaaaataggcctaccagttttattcaaagcatctgacatggccttgtaacggtggtaagaaacgtctggtgtaccaaattgacctttattgtaacaattatcatacttcaagtagtcaacgcggttatttgcaaagaattgagcatcttcttcctcacgccccagagacccagggtacccagcacaggtgtactcaccagcagacgaatacataccgaaaagaaagctgttattatgcaggtggtctgcaacatggcccataccgttgggaaatttgtgcttgtctgcaacgaggaaaccgtcggaatccctgccgctagaccaacagtcatctaggatgacatacttgtaacccatatcctttagccccaagtcagaaattctatcagcagtgtctagaagtagctgttcactgacatcgcaggcaaacgtattccagctgtcccaacccatctgtggggtgagaccaagaccattgtaactcggagaaactccgaaaacacccttcaaagtggtgcatgcggtgagaaagtagaaagcaaacatgaattctttgatttagtgtttgtgtgttgataagcagttgcttggttttttatgaaaaatagctagaaggaataagggattacaagagagatgttacaagaaagaagtaaaataaatttgattaatattgccattatcaaaagctatttatatgttgaaatcgtggagatcatgtgtgccagaaaaggccacagtttccggggagaggcataccttgaggtggctaggaatcacggagacctcttgacttgcagggtaggctagctagaattaagtgaggtgacaaggtttccatacagttttgaccttgagacgttgctacttacgatttgcagtatgcaagtctcatgctgcaaacaaaagaggaccgctcaggtaatcgctcaattagtggacgttatcaggggcgggagaggcgaaagtggtttttggtggtgtaagtaaaggtcgtccaaatatgcaggtgtttgggtgctatcctagtggaagctcggatcagtagataacccgcctagaagactagtggatcgatccccatcacctaataacttcgtatagcatacattatacgaagttatattaagggttctcgactctagaggatccgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctctagacctaataacttcgtatagcatacattatacgaagttatattaagggttgtcgacctgcagcgtacgaagcttcagctggcggccgctctagaactagagcggccgcgtgacagaatatgccaaagaacccataaataaatatgatataagagcgcccactgggccggcgttggtcagaggtgtggataaaccaatgaaaagacctgtaccaatagtaccaccaagggcaatcataccaatatgtctttgcttaagctctctcttcacttcagcgttctgtacttctccttcatcttcatcacctatgccatcctccatagagaacgtatcctcgccatttactctcgtcgggaaagagcgcaatggatacaattctttacttttctcatctttcaatggtattgacccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccaataacggaatccaactgggccggtaaccccagcaccacaaaccatacaaaaacagtatattagaaacccgataatggctaaaactttgatggaagcgacccagaactcgaattcaccgtaatatttgacagggaacaagttcattattgtgataattacccaaaaaatactaatccatgccgccagtggaactttgtacgtccaaaattgaatgacttggaaattatatcacactaattttcctcctgtggtagccctaagaacttttggtttgaatctgacgaataagatcggtccagttaagaatatgatcattgacacattaggaggaaatgagaaatgagaggtatgtaaatagaaatagactagctccacttttaagaattatttatgcaattaaatacatgggtgaccaaaagagcgggcggatacacgcgtcaccacaagcagaataaaaggtaaacctgaaattgttttaacataaaatgaaaaatgcttgtttgcaaccctatatagaatcataaaacattcgtgactataaaatgaataaactaaactattctaagaaaatgaaataaatgacaaaaaaacgtgttttttggactagaaggcttaatcaaaagctctcgagggcgcgccttaagccgagggtctcaacctatagaaggcgataccatgagctggaacagttgtgttaagtatagcatttggagaaagactaccaattttctggccaaaca

SEQ ID NO: 24 - DNA, 8316 bp, Artificial Sequence
  pMIE-024B
catcaactgtataatcagcttttggatctaaaccttgcaatctaacccatggaactgcatggttaacgtttggttgaacttggaagtaaaacaaaacagcttgtgcaccatcttgagaaacaaataaagctgctggccattgtgaatcttgtggtaatctcaatctgtacaagtcaccgttcaagatgattgggttaaccttttcagccaatgctaacaattctggaacaatttcgtcaccttcaacagtagctggatctaattccaaaccaaaagaaccacccatcatagcaacatgagctctaaaagtaaaaggaacagttctacctgtttgagcatttggaactgctgacaaatgagcacccattgcagatggtgggtaagctaatgaagtaccaaattgaattgtgattctatcgattgcatcagtgttatcagatgtccaaatttgtggaacatattgtaacatgccagcatcaaatctaccaccaccagaagcacaaccttcccataaaacatctgggaatcttgtagtcaaagtatcgaaaactctgtataaacccaacatgtattgatgatcagttgatggagatggtgtttcatgaatacctctgttgttatcccacttaacgtaagagataccagtatcttgcaacaaatttgtcatgaaatcgatgatgaaatcttgaacagctggtaatgccaaattcaaaaccaattggtttcttctttcagttcttgggtatggaccagcatgtaatgcccattctggatgttcatggtacaatgtagaatttgggttaaccatttctggttcaacccaaataccgaatctcaacttagttgattctgtaccgttaacagtcaaatttgtgatatcttgaacaactggtgtcaaaccatctggaaatctagctggatttggcatccagtcacccaaaccagcattatcagaaactcttggatatttgtcaccaaaccaaccatcatccataacgaacaaatgaacacccaaagctgcagattcttctgctaaagtttcgattgaagattggttgtaatcgaagtaaacaccttcccatgaatttaacaaaactggtctatctgatgtagcgaacttagacttcatcaaatggtttctgtacaatctatggaactttcttgaaacagaacccaaacctttatcagaataaacagcaacacattctggtgatgttaaagtttcacctggacccaaattccaagacaattgatctggattaaaacctaacaaagctctagtaaaaccttgtgaacctttttcaacttgtgctgaaaaagaacctgtgtaaaccaaattaaaaccccaagcttcaccttgagattctgtagtagctggatcaacgattgctaaaaatgggttatgcaaatgtgaagagtaaccagttgaagaaccaaaaccttgaacaccatattgaacctttgatctttgaacgttagcttctcttgcccagtcacctcttaatgaaaccatatccaaatcttcgtatgggaaatcgattgagatagaagccaatgcttcgatagtgatgttgcctggaccttggtttgtaacgttaactgatctaacgatagcatcgtactttgggaagattgagtaagacaaatcagctgcaactgaagagtagttatcgtacaaatgaacaaccaatgtagtagcatcttgtgcgtcaccaaaagtagctggtaaacctggcaatgcgtatttaccctcaataacttcatgagaaacgtattgcaaatctgaaactgtataaccagcagattctctaattctaactgctggtattctaaagtcacctctaccttgatctggaaattctcttctaattctacctggcataccaacccaaccattaacagctggttcaactggtgatggaatagtaccagaaacaacaccaccaaaatgatcagaaatcaagtcaccagttgaagaattaacatggaatctgtaagaaacgttgtcaccgttcaaagcgaatgaagtaccgtttgtaacgatagtttgtgaattagaagcaccaattgctggagctgcagctgaatgaccatataaagtaaacaaacccaaagcaacaactgcatgtgaagaaccaatcatgaattctttgatttagtgtttgtgtgttgataagcagttgcttggttttttatgaaaaatagctagaaggaataagggattacaagagagatgttacaagaaagaagtaaaataaatttgattaatattgccattatcaaaagctatttatatgttgaaatcgtggagatcatgtgtgccagaaaaggccacagtttccggggagaggcataccttgaggtggctaggaatcacggagacctcttgacttgcagggtaggctagctagaattaagtgaggtgacaaggtttccatacagttttgaccttgagacgttgctacttacgatttgcagtatgcaagtctcatgctgcaaacaaaagaggaccgctcaggtaatcgctcaattagtggacgttatcaggggcgggagaggcgaaagtggtttttggtggtgtaagtaaaggtcgtccaaatatgcaggtgtttgggtgctatcctagtggaagctcggatcagtagataacccgcctagaagactagtggatcgatccccatcacctaataacttcgtatagcatacattatacgaagttatattaagggttctcgactctagaggatccgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctctagacctaataacttcgtatagcatacattatacgaagttatattaagggttgtcgacctgcagcgtacgaagcttcagctggcggccgctctagaactagagcggccgcgtgacagaatatgccaaagaacccataaataaatatgatataagagcgcccactgggccggcgttggtcagaggtgtggataaaccaatgaaaagacctgtaccaatagtaccaccaagggcaatcataccaatatgtctttgcttaagctctctcttcacttcagcgttctgtacttctccttcatcttcatcacctatgccatcctccatagagaacgtatcctcgccatttactctcgtcgggaaagagcgcaatggatacaattctttacttttctcatctttcaatggtattgacccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccaataacggaatccaactgggccggtaaccccagcaccacaaaccatacaaaaacagtatattagaaacccgataatggctaaaactttgatggaagcgacccagaactcgaattcaccgtaatatttgacagggaacaagttcattattgtgataattacccaaaaaatactaatccatgccgccagtggaactttgtacgtccaaaattgaatgacttggaaattatatcacactaattttcctcctgtggtagccctaagaacttttggtttgaatctgacgaataagatcggtccagttaagaatatgatcattgacacattaggaggaaatgagaaatgagaggtatgtaaatagaaatagactagctccacttttaagaattatttatgcaattaaatacatgggtgaccaaaagagcgggcggatacacgcgtcaccacaagcagaataaaaggtaaacctgaaattgttttaacataaaatgaaaaatgcttgtttgcaaccctatatagaatcataaaacattcgtgactataaaatgaataaactaaactattctaagaaaatgaaataaatgacaaaaaaacgtgttttttggactagaaggcttaatcaaaagctctcgagggcgcgccttattgtctttccaagaaaacaactttagaaccgtattcagtatcgaatgagtattgtaaacccaaattcattaaagttgcaccagagtatgtttggtcac

SEQ ID NO: 25 - DNA, 8307 bp, Artificial Sequence
  pMIE-025B
tattgcttttcgatcatcaaaacatgagaatcgtagtcaccctcaaaagtcaattgaatacccatgttcattaatgttgaaccagaaacagtttggttgttatcaactctgtactttgctgaagcatctaaaccttgcaatctgaaccatggccaagagttattaattgttgctctagtttggaaagcgaacaaaacaacctttttaccatcttctgaaatgaattgacctgctgggtaatttgtttcttctggtaaagccaatctgtagaagtcaccagtgataacgattggattaatcttttcagacaattcgatcaaacctgggatttgttctctttcttctggttccaaatctgatggatctaattcaacaccaaaagaaccacccatcattgcaacatgagctctaaacttgactgatgtgattctttgagtgttaccgtttggaacatgagacaaatgtgcacccatagctgatggtgggtaagctaaagaagtaccgaattggattgcgattctttcaacagcatctgtatcatcagaagtccaaatttgtggaaaccattgcaaaacacctggatcaaatctaccaccaccagaagcacaaccttcccacaaaacatctgggaatcttgaagttaaagtttcgaaaacatggtataaacccaacatgtacttgtagtttaatgttggatctggagtttcatgaatacctctgttgttatcccacttaacgtatgagattggagattctctcaaaatttttgaaacagaatcgataataaactcttgaacttctggcaaagcaacattcaaaaccaattggtttcttgtttcagttcttgggtatgaacctgcatgaatagcccaatctggatgcttatcgtacaaatcagactttgggttaaccatttctggttcgaaccagataccgaacttcaaatcatcagatgcgttagcgattctcaattctgtgatgttttcaaccaatggagttaaaccatctggaaatctttccttatttggttgccagtcacccaaaccagctgaatcgttaactcttgggtacttattaccgaaccaaccatcatccataacgaacaatttaatacccaaatctgcagattgcttagcaatcttttcgatagctgtttcattaatttcgaaacctaaaccttcccatgaatttaacaaaactggtctagtttcttctgcgtacttagacttcatcaaatgctttctgtataatctatggaattgtcttgacataccaccaacacccttattagagaaaacagaaacaacttctggagtagtaaaagtttgacctggtttcaatggccatgagaaatccaaagaattaacacctaaaatagctctagtcaaaccttgtgaagatttttcaacatcaactgcaaatgaacctgtgtaaaccaaagaaaaaccccaagcttcaccttgagtttctgtagttgtagatgcaactaaagccaaaaatgggttatgcaaatgagaagaataacctgttgaagattgaaaaccttgagtaccaaaatcaacttttcttctaactctcatgccttctcttgcccagtcacctctgatttcgatcaaatccaaattatcttgttgcaaatcaacagaccatgaagaaactctcttcaaattaactgtagcgttaccacggttagtgatgttaactgatctaacaactgcatcgtactttgggaagattgagtaagacaaatcaacagcgattgaagagtagttatcgtacattctaacaactaatgttgaaacatcatcagcttcaccaaaagtagatggtaaacctggcaaacctggtttaccttcaacaacttcatgtgatttgtatctgaaatctgtaacagttgtaccagatgcttgttgcaattgaaaagctggtattctaaagtcacctctaccatgatctggaaattctcttctaactctaccgatcaaattaacccaaccttgaattggaccaatttctgcagtaataccatcttcagcaactggaccaccgtaatgatcattaatcaagtcaccagttgtgttatcaacatggaatctgtatgaaacgttgtcaccgttcaaagcgaaagatgtaccatcaacatgaattggatctgaagattctgccaaagcagagattgcttgacaaaataaaacagcagttgtagtgatacctcttaaagtaaccaaaaccatgaattctttgatttagtgtttgtgtgttgataagcagttgcttggttttttatgaaaaatagctagaaggaataagggattacaagagagatgttacaagaaagaagtaaaataaatttgattaatattgccattatcaaaagctatttatatgttgaaatcgtggagatcatgtgtgccagaaaaggccacagtttccggggagaggcataccttgaggtggctaggaatcacggagacctcttgacttgcagggtaggctagctagaattaagtgaggtgacaaggtttccatacagttttgaccttgagacgttgctacttacgatttgcagtatgcaagtctcatgctgcaaacaaaagaggaccgctcaggtaatcgctcaattagtggacgttatcaggggcgggagaggcgaaagtggtttttggtggtgtaagtaaaggtcgtccaaatatgcaggtgtttgggtgctatcctagtggaagctcggatcagtagataacccgcctagaagactagtggatcgatccccatcacctaataacttcgtatagcatacattatacgaagttatattaagggttctcgactctagaggatccgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctctagacctaataacttcgtatagcatacattatacgaagttatattaagggttgtcgacctgcagcgtacgaagcttcagctggcggccgctctagaactagagcggccgcgtgacagaatatgccaaagaacccataaataaatatgatataagagcgcccactgggccggcgttggtcagaggtgtggataaaccaatgaaaagacctgtaccaatagtaccaccaagggcaatcataccaatatgtctttgcttaagctctctcttcacttcagcgttctgtacttctccttcatcttcatcacctatgccatcctccatagagaacgtatcctcgccatttactctcgtcgggaaagagcgcaatggatacaattctttacttttctcatctttcaatggtattgacccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccaataacggaatccaactgggccggtaaccccagcaccacaaaccatacaaaaacagtatattagaaacccgataatggctaaaactttgatggaagcgacccagaactcgaattcaccgtaatatttgacagggaacaagttcattattgtgataattacccaaaaaatactaatccatgccgccagtggaactttgtacgtccaaaattgaatgacttggaaattatatcacactaattttcctcctgtggtagccctaagaacttttggtttgaatctgacgaataagatcggtccagttaagaatatgatcattgacacattaggaggaaatgagaaatgagaggtatgtaaatagaaatagactagctccacttttaagaattatttatgcaattaaatacatgggtgaccaaaagagcgggcggatacacgcgtcaccacaagcagaataaaaggtaaacctgaaattgttttaacataaaatgaaaaatgcttgtttgcaaccctatatagaatcataaaacattcgtgactataaaatgaataaactaaactattctaagaaaatgaaataaatgacaaaaaaacgtgttttttggactagaaggcttaatcaaaagctctcgagggcgcgcct

SEQ ID NO: 26 - DNA, 7518 bp, Artificial Sequence
  pMIE-026A
ctaaatcaaagaattcatgggtttgtcttcattgttattgtcagctgcattgttcggtttaaaagcatctgctttgaacaacggtttagcaagaactccacaaatgggttggaatacatggaactcttttgcttgtgaattaaacgaaactgttattttgaacgctgcagaaagaatcgtttctttgggttttagagatttgggttacgaatacgttgttttagatgattgttggtcagcaggtagaaattcttcaggttatttgatcgctgattctgaaaagttcccaaacggtatcgcacatttggctgataaagttcatgaattgggtttaaagatcggtatatattcttctgcaggtacttggacatgtgctagatatgaaggttcattgggttacgaagaaaaagatgctgcattatgggcatcttggggtatcgactatttgaagtacgataactgttacaacgaaggtgaagaaggtactccaaagttgtcattcgatagatacaacgctatgttcaaagcattgaatgctacaggtagaccaatgttgtactctttgtgtaactggggtgttgatggtccatggaattttgcaccaactattgctaattcatggagaacagctggtgacttgtctaatgtttgggatagagatgatgttaattgtccatgttctgaattggaaggtttagattgtaaaactccaggttacaagtgttcaatcatgaacgttttgaataaggcagtttattacccatctaaagcaattccaggtgcttggaacgatttggatatgttgcaagttggtaacggtggtttaacagatgatgaatcaatcgctcatatgtctttgtgggctgcattgaaatcaccattattgatgactaacgttatgacaaagatcgatccaccaactttgtctatcttgcaaaacccagcagttttagctgtttcacaagatccagttgcttctacaccagttagacaatggagatacttcgttgatgatgttgatgaaaacggtaaaggtgaaatccaaatgtactctggtccattgtctggtggtgaccaattggttttgttgttgaacgcaggttcaaaggctagagaaatgaacgctactttggttgatattttctgggaatctggtccaaaaggtacagcaaagcaagttaagcaacattgggatgtttacgatttgtgggctaatagaatgtctaatgaagatgctgcagctattattaacggtacttttacaggtccatcaccatataatttgactgcaatgggtggtgctcatgaagtttattcaagaccattgccatctaattcaaaggttttgatgggttctaaagttggttcagttcaaccatctggtactgttacagctcatgttagaccacatggtattgcaatgttgagattaagagctacagataagaaagatgaattataaggcgcgccctcgagagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcctaatgtgtcaatgatcatattcttaactggaccgatcttattcgtcagattcaaaccaaaagttcttagggctaccacaggaggaaaattagtgtgatataatttatcacctaataacttcgtatagcatacattatacgaagttatattaagggttctcgactctagaggatccgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctctagacctaataacttcgtatagcatacattatacgaagttatattaagggttgtcgacctgcagcgtacgaagcttcagctggcggccgctctagaactagagcggccgcgtgacagaatatgccaaagaacccataaataaatatgatataagagcgcccactgggccggcgttggtcagaggtgtggataaaccaatgaaaagacctgtaccaatagtaccaccaagggcaatcataccaatatgtctttgcttaagctctctcttcacttcagcgttctgtacttctccttcatcttcatcacctatgccatcctccatagagaacgtatcctcgccatttactctcgtcgggaaagagcgcaatggatacaattctttacttttctcatctttcaatggtattgacccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccaataacggaatccaactgggccggtaaccccagcaccacaaaccatacaaaaacagtatattagaaacccgataatggctaaaactttgatggaagcgacccagaactcgaattcaccgtaatatttgacagggaacaagttcattattgtgataattacccaaaaaatactaatccatgccgccagtggaactttgtacgtccaaaattgaatgacttggggggatcgatccactagtcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaaca

SEQ ID NO: 27 - DNA, 8402 bp, Artificial Sequence
  pMIE-031, can1::AnGAL Hyg
catcaactgtataatcagcttttggatctaaaccttgcaatctaacccatggaactgcatggttaacgtttggttgaacttggaagtaaaacaaaacagcttgtgcaccatcttgagaaacaaataaagctgctggccattgtgaatcttgtggtaatctcaatctgtacaagtcaccgttcaagatgattgggttaaccttttcagccaatgctaacaattctggaacaatttcgtcaccttcaacagtagctggatctaattccaaaccaaaagaaccacccatcatagcaacatgagctctaaaagtaaaaggaacagttctacctgtttgagcatttggaactgctgacaaatgagcacccattgcagatggtgggtaagctaatgaagtaccaaattgaattgtgattctatcgattgcatcagtgttatcagatgtccaaatttgtggaacatattgtaacatgccagcatcaaatctaccaccaccagaagcacaaccttcccataaaacatctgggaatcttgtagtcaaagtatcgaaaactctgtataaacccaacatgtattgatgatcagttgatggagatggtgtttcatgaatacctctgttgttatcccacttaacgtaagagataccagtatcttgcaacaaatttgtcatgaaatcgatgatgaaatcttgaacagctggtaatgccaaattcaaaaccaattggtttcttctttcagttcttgggtatggaccagcatgtaatgcccattctggatgttcatggtacaatgtagaatttgggttaaccatttctggttcaacccaaataccgaatctcaacttagttgattctgtaccgttaacagtcaaatttgtgatatcttgaacaactggtgtcaaaccatctggaaatctagctggatttggcatccagtcacccaaaccagcattatcagaaactcttggatatttgtcaccaaaccaaccatcatccataacgaacaaatgaacacccaaagctgcagattcttctgctaaagtttcgattgaagattggttgtaatcgaagtaaacaccttcccatgaatttaacaaaactggtctatctgatgtagcgaacttagacttcatcaaatggtttctgtacaatctatggaactttcttgaaacagaacccaaacctttatcagaataaacagcaacacattctggtgatgttaaagtttcacctggacccaaattccaagacaattgatctggattaaaacctaacaaagctctagtaaaaccttgtgaacctttttcaacttgtgctgaaaaagaacctgtgtaaaccaaattaaaaccccaagcttcaccttgagattctgtagtagctggatcaacgattgctaaaaatgggttatgcaaatgtgaagagtaaccagttgaagaaccaaaaccttgaacaccatattgaacctttgatctttgaacgttagcttctcttgcccagtcacctcttaatgaaaccatatccaaatcttcgtatgggaaatcgattgagatagaagccaatgcttcgatagtgatgttgcctggaccttggtttgtaacgttaactgatctaacgatagcatcgtactttgggaagattgagtaagacaaatcagctgcaactgaagagtagttatcgtacaaatgaacaaccaatgtagtagcatcttgtgcgtcaccaaaagtagctggtaaacctggcaatgcgtatttaccctcaataacttcatgagaaacgtattgcaaatctgaaactgtataaccagcagattctctaattctaactgctggtattctaaagtcacctctaccttgatctggaaattctcttctaattctacctggcataccaacccaaccattaacagctggttcaactggtgatggaatagtaccagaaacaacaccaccaaaatgatcagaaatcaagtcaccagttgaagaattaacatggaatctgtaagaaacgttgtcaccgttcaaagcgaatgaagtaccgtttgtaacgatagtttgtgaattagaagcaccaattgctggagctgcagctgaatgaccatataaagtaaacaaacccaaagcaacaactgcatgtgaagaaccaatcatgaattctttgatttagtgtttgtgtgttgataagcagttgcttggttttttatgaaaaatagctagaaggaataagggattacaagagagatgttacaagaaagaagtaaaataaatttgattaatattgccattatcaaaagctatttatatgttgaaatcgtggagatcatgtgtgccagaaaaggccacagtttccggggagaggcataccttgaggtggctaggaatcacggagacctcttgacttgcagggtaggctagctagaattaagtgaggtgacaaggtttccatacagttttgaccttgagacgttgctacttacgatttgcagtatgcaagtctcatgctgcaaacaaaagaggaccgctcaggtaatcgctcaattagtggacgttatcaggggcgggagaggcgaaagtggtttttggtggtgtaagtaaaggtcgtccaaatatgcaggtgtttgggtgctatcctagtggaagctcggatcagtagataacccgcctagaagactagtggatctgatatcacctaataacttcgtatagcatacattatacgaagttatattaagggttctcgagagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattactactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctctagacctaataacttcgtatagcatacattatacgaagttatattaagggttgtcgacctgcagcgtacgaagcttcagctggcggccgcgtgacagaatatgccaaagaacccataaataaatatgatataagagcgcccactgggccggcgttggtcagaggtgtggataaaccaatgaaaagacctgtaccaatagtaccaccaagggcaatcataccaatatgtctttgcttaagctctctcttcacttcagcgttctgtacttctccttcatcttcatcacctatgccatcctccatagagaacgtatcctcgccatttactctcgtcgggaaagagcgcaatggatacaattctttacttttctcatctttcaatggtattgacccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccaataacggaatccaactgggccggtaaccccagcaccacaaaccatacaaaaacagtatattagaaacccgataatggctaaaactttgatggaagcgacccagaactcgaattcaccgtaatatttgacagggaacaagttcattattgtgataattacccaaaaaatactaatccatgccgccagtggaactttgtacgtccaaaattgaatgacttggaaattatatcacactaattttcctcctgtggtagccctaagaacttttggtttgaatctgacgaataagatcggtccagttaagaatatgatcattgacacattaggaggaaatgagaaatgagaggtatgtaaatagaaatagactagctccacttttaagaattatttatgcaattaaatacatgggtgaccaaaagagcgggcggatacacgcgtcaccacaagcagaataaaaggtaaacctgaaattgttttaacataaaatgaaaaatgcttgtttgcaaccctatatagaatcataaaacattcgtgactataaaatgaataaactaaactattctaagaaaatgaaataaatgacaaaaaaacgtgttttttggactagaaggcttaatcaaaagctctcgagggcgcgccttattgtctttccaagaaaacaactttagaaccgtattcagtatcgaatgagtattgtaaacccaaattcattaaagttgcaccagagtatgtttggtcac

SEQ ID NO: 28 - DNA, 8393 bp, Artificial Sequence
  pMIE-032, can1::GibGAL Hyg
tatcaactctgtactttgctgaagcatctaaaccttgcaatctgaaccatggccaagagttattaattgttgctctagtttggaaagcgaacaaaacaacctttttaccatcttctgaaatgaattgacctgctgggtaatttgtttcttctggtaaagccaatctgtagaagtcaccagtgataacgattggattaatcttttcagacaattcgatcaaacctgggatttgttctctttcttctggttccaaatctgatggatctaattcaacaccaaaagaaccacccatcattgcaacatgagctctaaacttgactgatgtgattctttgagtgttaccgtttggaacatgagacaaatgtgcacccatagctgatggtgggtaagctaaagaagtaccgaattggattgcgattctttcaacagcatctgtatcatcagaagtccaaatttgtggaaaccattgcaaaacacctggatcaaatctaccaccaccagaagcacaaccttcccacaaaacatctgggaatcttgaagttaaagtttcgaaaacatggtataaacccaacatgtacttgtagtttaatgttggatctggagtttcatgaatacctctgttgttatcccacttaacgtatgagattggagattctctcaaaatttttgaaacagaatcgataataaactcttgaacttctggcaaagcaacattcaaaaccaattggtttcttgtttcagttcttgggtatgaacctgcatgaatagcccaatctggatgcttatcgtacaaatcagactttgggttaaccatttctggttcgaaccagataccgaacttcaaatcatcagatgcgttagcgattctcaattctgtgatgttttcaaccaatggagttaaaccatctggaaatctttccttatttggttgccagtcacccaaaccagctgaatcgttaactcttgggtacttattaccgaaccaaccatcatccataacgaacaatttaatacccaaatctgcagattgcttagcaatcttttcgatagctgtttcattaatttcgaaacctaaaccttcccatgaatttaacaaaactggtctagtttcttctgcgtacttagacttcatcaaatgctttctgtataatctatggaattgtcttgacataccaccaacacccttattagagaaaacagaaacaacttctggagtagtaaaagtttgacctggtttcaatggccatgagaaatccaaagaattaacacctaaaatagctctagtcaaaccttgtgaagatttttcaacatcaactgcaaatgaacctgtgtaaaccaaagaaaaaccccaagcttcaccttgagtttctgtagttgtagatgcaactaaagccaaaaatgggttatgcaaatgagaagaataacctgttgaagattgaaaaccttgagtaccaaaatcaacttttcttctaactctcatgccttctcttgcccagtcacctctgatttcgatcaaatccaaattatcttgttgcaaatcaacagaccatgaagaaactctcttcaaattaactgtagcgttaccacggttagtgatgttaactgatctaacaactgcatcgtactttgggaagattgagtaagacaaatcaacagcgattgaagagtagttatcgtacattctaacaactaatgttgaaacatcatcagcttcaccaaaagtagatggtaaacctggcaaacctggtttaccttcaacaacttcatgtgatttgtatctgaaatctgtaacagttgtaccagatgcttgttgcaattgaaaagctggtattctaaagtcacctctaccatgatctggaaattctcttctaactctaccgatcaaattaacccaaccttgaattggaccaatttctgcagtaataccatcttcagcaactggaccaccgtaatgatcattaatcaagtcaccagttgtgttatcaacatggaatctgtatgaaacgttgtcaccgttcaaagcgaaagatgtaccatcaacatgaattggatctgaagattctgccaaagcagagattgcttgacaaaataaaacagcagttgtagtgatacctcttaaagtaaccaaaaccatgaattctttgatttagtgtttgtgtgttgataagcagttgcttggttttttatgaaaaatagctagaaggaataagggattacaagagagatgttacaagaaagaagtaaaataaatttgattaatattgccattatcaaaagctatttatatgttgaaatcgtggagatcatgtgtgccagaaaaggccacagtttccggggagaggcataccttgaggtggctaggaatcacggagacctcttgacttgcagggtaggctagctagaattaagtgaggtgacaaggtttccatacagttttgaccttgagacgttgctacttacgatttgcagtatgcaagtctcatgctgcaaacaaaagaggaccgctcaggtaatcgctcaattagtggacgttatcaggggcgggagaggcgaaagtggtttttggtggtgtaagtaaaggtcgtccaaatatgcaggtgtttgggtgctatcctagtggaagctcggatcagtagataacccgcctagaagactagtggatctgatatcacctaataacttcgtatagcatacattatacgaagttatattaagggttctcgagagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattactactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctctagacctaataacttcgtatagcatacattatacgaagttatattaagggttgtcgacctgcagcgtacgaagcttcagctggcggccgcgtgacagaatatgccaaagaacccataaataaatatgatataagagcgcccactgggccggcgttggtcagaggtgtggataaaccaatgaaaagacctgtaccaatagtaccaccaagggcaatcataccaatatgtctttgcttaagctctctcttcacttcagcgttctgtacttctccttcatcttcatcacctatgccatcctccatagagaacgtatcctcgccatttactctcgtcgggaaagagcgcaatggatacaattctttacttttctcatctttcaatggtattgacccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccaataacggaatccaactgggccggtaaccccagcaccacaaaccatacaaaaacagtatattagaaacccgataatggctaaaactttgatggaagcgacccagaactcgaattcaccgtaatatttgacagggaacaagttcattattgtgataattacccaaaaaatactaatccatgccgccagtggaactttgtacgtccaaaattgaatgacttggaaattatatcacactaattttcctcctgtggtagccctaagaacttttggtttgaatctgacgaataagatcggtccagttaagaatatgatcattgacacattaggaggaaatgagaaatgagaggtatgtaaatagaaatagactagctccacttttaagaattatttatgcaattaaatacatgggtgaccaaaagagcgggcggatacacgcgtcaccacaagcagaataaaaggtaaacctgaaattgttttaacataaaatgaaaaatgcttgtttgcaaccctatatagaatcataaaacattcgtgactataaaatgaataaactaaactattctaagaaaatgaaataaatgacaaaaaaacgtgttttttggactagaaggcttaatcaaaagctctcgagggcgcgccttattgcttttcgatcatcaaaacatgagaatcgtagtcaccctcaaaagtcaattgaatacccatgttcattaatgttgaaccagaaacagtttggttgt

SEQ ID NO: 29 - DNA, 7604 bp, Artificial Sequence
  pMIE-034, can1::AfGAL Hyg
ctttagaacccatcaaaacctttgaattagatggcaatggtcttgaataaacttcatgagcaccacccattgcagtcaaattatatggtgatggacctgtaaaagtaccgttaataatagctgcagcatcttcattagacattctattagcccacaaatcgtaaacatcccaatgttgcttaacttgctttgctgtaccttttggaccagattcccagaaaatatcaaccaaagtagcgttcatttctctagcctttgaacctgcgttcaacaacaaaaccaattggtcaccaccagacaatggaccagagtacatttggatttcacctttaccgttttcatcaacatcatcaacgaagtatctccattgtctaactggtgtagaagcaactggatcttgtgaaacagctaaaactgctgggttttgcaagatagacaaagttggtggatcgatctttgtcataacgttagtcatcaataatggtgatttcaatgcagcccacaaagacatatgagcgattgattcatcatctgttaaaccaccgttaccaacttgcaacatatccaaatcgttccaagcacctggaattgctttagatgggtaataaactgccttattcaaaacgttcatgattgaacacttgtaacctggagttttacaatctaaaccttccaattcagaacatggacaattaacatcatctctatcccaaacattagacaagtcaccagctgttctccatgaattagcaatagttggtgcaaaattccatggaccatcaacaccccagttacacaaagagtacaacattggtctacctgtagcattcaatgctttgaacatagcgttgtatctatcgaatgacaactttggagtaccttcttcaccttcgttgtaacagttatcgtacttcaaatagtcgataccccaagatgcccataatgcagcatctttttcttcgtaacccaatgaaccttcatatctagcacatgtccaagtacctgcagaagaatatataccgatctttaaacccaattcatgaactttatcagccaaatgtgcgataccgtttgggaacttttcagaatcagcgatcaaataacctgaagaatttctacctgctgaccaacaatcatctaaaacaacgtattcgtaacccaaatctctaaaacccaaagaaacgattctttctgcagcgttcaaaataacagtttcgtttaattcacaagcaaaagagttccatgtattccaacccatttgtggagttcttgctaaaccgttgttcaaagcagatgcttttaaaccgaacaatgcagctgacaataacaatgaagacaaacccatgaattctttgatttagtgtttgtgtgttgataagcagttgcttggttttttatgaaaaatagctagaaggaataagggattacaagagagatgttacaagaaagaagtaaaataaatttgattaatattgccattatcaaaagctatttatatgttgaaatcgtggagatcatgtgtgccagaaaaggccacagtttccggggagaggcataccttgaggtggctaggaatcacggagacctcttgacttgcagggtaggctagctagaattaagtgaggtgacaaggtttccatacagttttgaccttgagacgttgctacttacgatttgcagtatgcaagtctcatgctgcaaacaaaagaggaccgctcaggtaatcgctcaattagtggacgttatcaggggcgggagaggcgaaagtggtttttggtggtgtaagtaaaggtcgtccaaatatgcaggtgtttgggtgctatcctagtggaagctcggatcagtagataacccgcctagaagactagtggatctgatatcacctaataacttcgtatagcatacattatacgaagttatattaagggttctcgagagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattactactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctctagacctaataacttcgtatagcatacattatacgaagttatattaagggttgtcgacctgcagcgtacgaagcttcagctggcggccgcgtgacagaatatgccaaagaacccataaataaatatgatataagagcgcccactgggccggcgttggtcagaggtgtggataaaccaatgaaaagacctgtaccaatagtaccaccaagggcaatcataccaatatgtctttgcttaagctctctcttcacttcagcgttctgtacttctccttcatcttcatcacctatgccatcctccatagagaacgtatcctcgccatttactctcgtcgggaaagagcgcaatggatacaattctttacttttctcatctttcaatggtattgacccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccaataacggaatccaactgggccggtaaccccagcaccacaaaccatacaaaaacagtatattagaaacccgataatggctaaaactttgatggaagcgacccagaactcgaattcaccgtaatatttgacagggaacaagttcattattgtgataattacccaaaaaatactaatccatgccgccagtggaactttgtacgtccaaaattgaatgacttggaaattatatcacactaattttcctcctgtggtagccctaagaacttttggtttgaatctgacgaataagatcggtccagttaagaatatgatcattgacacattaggaggaaatgagaaatgagaggtatgtaaatagaaatagactagctccacttttaagaattatttatgcaattaaatacatgggtgaccaaaagagcgggcggatacacgcgtcaccacaagcagaataaaaggtaaacctgaaattgttttaacataaaatgaaaaatgcttgtttgcaaccctatatagaatcataaaacattcgtgactataaaatgaataaactaaactattctaagaaaatgaaataaatgacaaaaaaacgtgttttttggactagaaggcttaatcaaaagctctcgagggcgcgccttataattcatctttcttatctgtagctcttaatctcaacattgcaataccatgtggtctaacatgagctgtaacagtaccagatggttgaactgaaccaa

SEQ ID NO: 30 - DNA, 89 bp, Artificial Sequence
  oligonucleotide 3ScPDC5 -210F
ctaatacgtaaacctgcattaaggtaagattatatcagaaaatgtgttgcaagaaatgcattatgttcgtacgctgcaggtcgacaacc

SEQ ID NO: 31 - DNA, 90 bp, Artificial Sequence
  oligonucleotide 6ScPDC5 stopR
tacacaaacgttgaatcatgagttttatgttaattagcttattgtttagcgttagtagcggcagtcggccactagtggatctgatatcac

SEQ ID NO: 32 - DNA, 88 bp, Artificial Sequence
  oligonucleotide 4ScPDC5 -136F
gattatgacaatctctcgaaagaaatttcatatgatgagacttgaataatgcagcggcgcttgcttcgtacgctgcaggtcgacaacc

SEQ ID NO: 33 - DNA, 6935 bp, Artificial Sequence
  pMIE-008, LDH into ScPDC1
atatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaaatcacgagcgacggtaatttctttctcgataagaggccacgtgctttatgagggtaacatcaattcaagaaggagggaaacacttcctttttctggccctgataatagtatgagggtgaagccaaaataaaggattcgcgcccaaatcggcatctttaaatgcaggtatgcgatagttcctcactctttccttactcacgtcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagtaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcccagtcttcgatgctccacaaaacttggttgaacaagctaagttgactgctgctaccaacgctaagcaataagcgatttaatctctaattattagttaaagttttataagcatttttatgtaacgaaaaataaattggttcatattattactgcactgtcactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacgcggccgccagctgcgattccgcccgctaaacgcatatttttgttgcctggtggcatttgcaaaatgcataacctatgcatttaaaagattatgtatgctcttctgacttttcgtgtgatggagctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccgtttttctgttagacggtgtcttgatctacttgctatcgttcaacaccaccttattttctaactattttttttttagctcatttgaatcagcttatggtgatggcacatttttgcataaacctagctgtcctcgttgaacatagggatccgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttggaagatcttcattgacgaaccttaacgccagtttctttgaaggccttgtccataaccttctttaattgatctgctgaagcagtcattaattcttgttccttatcgcttaatggcatttcgataacttgttcaagacccttgcggccaacaacagcaggagtaccgatgtgaagatcgtgtaaaccatattcaccatccattggtactgaaagtggaagtacacggtgttcatcgttcaagatagccttagcgatcattgctgaagcagtaccgataccgtagaaggtagcacctttcttgttaataatgtcgtaagccatgtccttaacttcttggtggatgtcttcaagcttagattcaggcatgttgtgagccttaacccagtcagcaacctttacgccagcaacattgttgtagctccatgctggaaattcagtatcaccgtgttcaccaagcatgtatgcattaactgaacttgggtcaacgttttccattttaccaataactttttgaagacgaccagtatcaagtgaagtacctgaaccgataacacgatccttagggaagcctgacattctccaagttgcgtgagttaagatatcaactgggttagcaactactaagaaaataccttcaaaacctgattcaacaactggttcaacgattgatgataaaatcttcaagttcttgttaacaagatcaagacgagtttcgcctggcttttgtggagcaccagcagtaataacaactaagtcagcatccttacaatctgggtagtcagctgcgtaaatgttctttggagaagtccaaggagttgcgtcagctaaatcgattgcgtcaccttcaacgtgttccttagcgatatcgataatacctaattcttcagcgataccttgttgtaccattgaaaatgcaaaggtagaacctacagcaccatcaccgactaaaataactttacgaggtttttcctctcttgccatggttttatatttgttgtaaaaagtagataattacttccttgatgatctgtaaaaaagagaaaaagaaagcatctaagaacttgaaaaactaagaattagaaaagaccaa

SEQ ID NO: 34 - protein, 532 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 35 - DNA, 1599 bp, Saccharomyces cerevisiae
atgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatag

SEQ ID NO: 36 - DNA, 8066 bp, Artificial Sequence
  pMIPk124
  misc_feature (1121)..(1121): n is a, c, g, or t
  misc_feature (4736)..(4736): n is a, c, g, or t
ttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacggatccgtatcatttgtagcccacgccacccggaaaaaccaccattgtcctcagcagtccgccaaaatatggatgcgctcaatcaactttccctcccccgtcaatgccaaaaggataacgacacactattaagagcgcatcatttgtaaaagccgaggaagggggatacgctaaccggagacgtctcgcctcactctcggagctgagccgccctccttaagaaattcatgggaagaacacccttcgcggcttctgaacggctcgccctcgtccattggtcacctcacagtggcaactaataaggacattatagcaatagaaattaaaatggtgcacagaaatacaataggatcgaataggataggatacaataagatacggaatattagactatactgtgatacggtacggtacgatacgctacgatacgatacgatagaggataccacggatataacgtagtattatttttcattattgggggtttttttctgtttgaattttccacgtcaagagtatcccatctgacaggaaccgatggactcgtcacagtacctatcgcccgagttcaatccatggacgcttcgggtgaaggatcttcgtccgctgttggcaagccatgggatcagggcgtcgccaagggacagaaaggcggatcttgtacgtctcttcaacacagagctgcgtccgaaacttactgagagtcttaacaccaataatcccaaaaacaacaacaacaatacagatactatagacactatagacactatagacactactaacanccctttaaagcgccgccgattaagcaatgttgatgagccgtcaattccatatactcagctggcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacgcggccgcgagctctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatgtcattaagcggctttagagttgatttcatcagataatttagcttgagcaaccaagatttctggagcatcgaattcatccaagaataattcaatgactctaatcttatcttccttgttgaatgcttcatccttcatcaaagcgtccaagtccttagcggatttaacaacatggttttcatattgggtcttgtcagcaaagagcttcaataacaattggtgatcccatggttgaatttggttgtagtcctcatgacgaccgtggatcaacttttcgatagtgtaacctctgttgtttaagatgaagatgtatggcttgatgttccatcttgcagcatctgagattgattggacagtcaattgtaaagaaccatcaccaataaacaaaacagttcttctttcttgttcgccagtttgtttgtgtgcatcttcagcagcaaatgcagcaccaactgcagctggtaaggagaaaccaatggaaccccataagacttgggagatagactttgaatctcttggtatgggtagccaagactagtcgatatcacctaataacttcgtatagcatacattatacgaagttatattaagggttctcgagaattcttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagtaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattacatactaggatctctagacctaataacttcgtatagcatacattatacgaagttatattaagggttgtcgacggatccttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgccaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatgggggggggcgaaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaaccatggcaagagaggaaaaacctcgtaaagttattttagtcggtgatggtgctgtaggttctacctttgcattttcaatggtacaacaaggtatcgctgaagaattaggtattatcgatatcgctaaggaacacgttgaaggtgacgcaatcgatttagctgacgcaactccttggacttctccaaagaacatttacgcagctgactacccagattgtaaggatgctgacttagttgttattactgctggtgctccacaaaagccaggcgaaactcgtcttgatcttgttaacaagaacttgaagattttatcatcaatcgttgaaccagttgttgaatcaggttttgaaggtattttcttagtagttgctaacccagttgatatcttaactcacgcaacttggagaatgtcaggcttccctaaggatcgtgttatcggttcaggtacttcacttgatactggtcgtcttcaaaaagttattggtaaaatggaaaacgttgacccaagttcagttaatgcatacatgcttggtgaacacggtgatactgaatttccagcatggagctacaacaatgttgctggcgtaaaggttgctgactgggttaaggctcacaacatgcctgaatctaagcttgaagacatccaccaagaagttaaggacatggcttacgacattattaacaagaaaggtgctaccttctacggtatcggtactgcttcagcaatgatcgctaaggctatcttgaacgatgaacaccgtgtacttccactttcagtaccaatggatggtgaatatggtttacacgatcttcacatcggtactcctgctgttgttggccgcaagggtcttgaacaagttatcgaaatgccattaagcgataaggaacaagaattaatgactgcttcagcagatcaattaaagaaggttatggacaaggccttcaaagaaactggcgttaaggttcgtcaatgaagatcttccaagctttggacttcttcgccagaggtttggtcaagtctccaatcaaggttgtcggcttgtctaccttgccagaaatttacgaaaagatggaaaagggtcaaatcgttggtagatacgttgttgacac

SEQ ID NO: 37 - DNA, 9073 bp, Artificial Sequence
  pEKOPA8
gcggccgccagctgtgtttgcagcctctacgttgagaataatgttcccacccccgggcgagacttctgcgggggggcatgagtgcgggaaacgccggagtccgttcggtggtattgttttacattttcgttattacccaacgggggctagtggtcttcaatgaggaagttctggaaattgtcggatagaaaatagtcccaattctattgttactaatggcgctagtgatattgttgcagtaaatggtcaattcccaattattccatacacggggtgcgtcggcgcgagggtagggtgatggttttcacctttggtgggcctcaatgtgagggtgggtggcatcatagtcaagataacaaattgtacaccaactcttataaaaggacacccatttgccgccatgtatattggttgatgtgtggatcctgatgctttactgtgatctctgatactctctgatactctttgatactcttttccatgcatgtttcgcttttccctcagctactgcttcacctcccctcccctcccctctcccccttctttctcccgtttctgtgtacaacttctttatagacccactaacccccaacactgtatttaacacatccccattgaccttcattgaccttcattgaccctcccccaccctttccctcccccaccagcgtatttcccttttctctccccattctctctgctcttctcggctcgttgtcgctcgcggtcattttttttcgcccttcttttcccgcttttcccgtagctggtgtagtccgaaactgtgctgatcttcttcctcatatgggaccatctgggtagagctcctctatttattatccgaccctattccaccttccttgcttggttgacaatttaagatgaagttcctcccatttcttttgtactccttttctcctctcttgtatttttgtctccttttcttgtttcttccctctgtaagccatccaaagaacagaacccatctttctcgtgctgcttaaactaaaccgaacccacacgcaatcttaaaagaaccattaattaaatggttttggttactttaagaggtatcactacaactgctgttttattttgtcaagcaatctctgctttggcagaatcttcagatccaattcatgttgatggtacatctttcgctttgaacggtgacaacgtttcatacagattccatgttgataacacaactggtgacttgattaatgatcattacggtggtccagttgctgaagatggtattactgcagaaattggtccaattcaaggttgggttaatttgatcggtagagttagaagagaatttccagatcatggtagaggtgactttagaataccagcttttcaattgcaacaagcatctggtacaactgttacagatttcagatacaaatcacatgaagttgttgaaggtaaaccaggtttgccaggtttaccatctacttttggtgaagctgatgatgtttcaacattagttgttagaatgtacgataactactcttcaatcgctgttgatttgtcttactcaatcttcccaaagtacgatgcagttgttagatcagttaacatcactaaccgtggtaacgctacagttaatttgaagagagtttcttcatggtctgttgatttgcaacaagataatttggatttgatcgaaatcagaggtgactgggcaagagaaggcatgagagttagaagaaaagttgattttggtactcaaggttttcaatcttcaacaggttattcttctcatttgcataacccatttttggctttagttgcatctacaactacagaaactcaaggtgaagcttggggtttttctttggtttacacaggttcatttgcagttgatgttgaaaaatcttcacaaggtttgactagagctattttaggtgttaattctttggatttctcatggccattgaaaccaggtcaaacttttactactccagaagttgtttctgttttctctaataagggtgttggtggtatgtcaagacaattccatagattatacagaaagcatttgatgaagtctaagtacgcagaagaaactagaccagttttgttaaattcatgggaaggtttaggtttcgaaattaatgaaacagctatcgaaaagattgctaagcaatctgcagatttgggtattaaattgttcgttatggatgatggttggttcggtaataagtacccaagagttaacgattcagctggtttgggtgactggcaaccaaataaggaaagatttccagatggtttaactccattggttgaaaacatcacagaattgagaatcgctaacgcatctgatgatttgaagttcggtatctggttcgaaccagaaatggttaacccaaagtctgatttgtacgataagcatccagattgggctattcatgcaggttcatacccaagaactgaaacaagaaaccaattggttttgaatgttgctttgccagaagttcaagagtttattatcgattctgtttcaaaaattttgagagaatctccaatctcatacgttaagtgggataacaacagaggtattcatgaaactccagatccaacattaaactacaagtacatgttgggtttataccatgttttcgaaactttaacttcaagattcccagatgttttgtgggaaggttgtgcttctggtggtggtagatttgatccaggtgttttgcaatggtttccacaaatttggacttctgatgatacagatgctgttgaaagaatcgcaatccaattcggtacttctttagcttacccaccatcagctatgggtgcacatttgtctcatgttccaaacggtaacactcaaagaatcacatcagtcaagtttagagctcatgttgcaatgatgggtggttcttttggtgttgaattagatccatcagatttggaaccagaagaaagagaacaaatcccaggtttgatcgaattgtctgaaaagattaatccaatcgttatcactggtgacttctacagattggctttaccagaagaaacaaattacccagcaggtcaattcatttcagaagatggtaaaaaggttgttttgttcgctttccaaactagagcaacaattaataactcttggccatggttcagattgcaaggtttagatgcttcagcaaagtacagagttgataacaaccaaactgtttctggttcaacattaatgaacatgggtattcaattgacttttgagggtgactacgattctcatgttttgatgatcgaaaagcaataattaattaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatttcttccaaaaaaaaaaccgatgaattagtggaaccaaggaaaaaaaaagaggtatccttgattaaggaacactgtttaaacagtgtggtttccaaaaccctgaaactgcattagtgtaatacaagactagacacctcgatacaaataatggttactcaattcaaaactgcctttgaaacatcatgaaaactgtttcaccctctgtgaagcataaacactagaaagccaatgaagagctctacaagcctcatatgggttcaatgggtctgcaatgaccgcatacgggcttggacaattaccttctattgaatttctgagaagagatacatctgaccagcaatgtaagcagacaatcccaattctgtaaacaacctctttgtccataattccccatcagaagagtgaaaaatgccctcaaaatgcatgcgccactcccacctctcaactgcactgcgccacctctgagggtcctttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttgcgttttcttttcaacttttttttactttttccttcaacagacaaatctaacttatatagatctatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagagatctgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattgatatcgtcaaggctgtccaagaggcaaccaatggtggtccacatggtgtcatcaatgtctctgtctctgaagctgcaatttctcaatcttgtgaatacgttagacctctaggtaaggttgttcttgttggtttaccagcaggcgcacaagtcaaaactggtgtctttgaagccgttgtcaagtctattgaaattaagggttcttatgtcggtaacagaaaggataccgccgaagcacttgacttctacactagaggcttggtcaagtctccattcaagattgtcggtttatccgaattgccaaaagtctttgaactcatggaacagggtaagattttaggtagaatggtcttagacacctccaaataagtctagagagtgtatacctccccgcttttgctgctactaattaatacccactattaatttccttctattacaaaacgcctctcagactcccacacacacacttacactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaac

SEQ ID NO: 38 - DNA, 9082 bp, Artificial Sequence
  pEKOPA9
gcggccgccagctgtgtttgcagcctctacgttgagaataatgttcccacccccgggcgagacttctgcgggggggcatgagtgcgggaaacgccggagtccgttcggtggtattgttttacattttcgttattacccaacgggggctagtggtcttcaatgaggaagttctggaaattgtcggatagaaaatagtcccaattctattgttactaatggcgctagtgatattgttgcagtaaatggtcaattcccaattattccatacacggggtgcgtcggcgcgagggtagggtgatggttttcacctttggtgggcctcaatgtgagggtgggtggcatcatagtcaagataacaaattgtacaccaactcttataaaaggacacccatttgccgccatgtatattggttgatgtgtggatcctgatgctttactgtgatctctgatactctctgatactctttgatactcttttccatgcatgtttcgcttttccctcagctactgcttcacctcccctcccctcccctctcccccttctttctcccgtttctgtgtacaacttctttatagacccactaacccccaacactgtatttaacacatccccattgaccttcattgaccttcattgaccctcccccaccctttccctcccccaccagcgtatttcccttttctctccccattctctctgctcttctcggctcgttgtcgctcgcggtcattttttttcgcccttcttttcccgcttttcccgtagctggtgtagtccgaaactgtgctgatcttcttcctcatatgggaccatctgggtagagctcctctatttattatccgaccctattccaccttccttgcttggttgacaatttaagatgaagttcctcccatttcttttgtactccttttctcctctcttgtatttttgtctccttttcttgtttcttccctctgtaagccatccaaagaacagaacccatctttctcgtgctgcttaaactaaaccgaacccacacgcaatcttaaaagaaccattaattaaatgattggttcttcacatgcagttgttgctttgggtttgtttactttatatggtcattcagctgcagctccagcaattggtgcttctaattcacaaactatcgttacaaacggtacttcattcgctttgaacggtgacaacgtttcttacagattccatgttaattcttcaactggtgacttgatttctgatcattttggtggtgttgtttctggtactattccatcaccagttgaaccagctgttaatggttgggttggtatgccaggtagaattagaagagaatttccagatcaaggtagaggtgactttagaataccagcagttagaattagagaatctgctggttatacagtttcagatttgcaatacgtttctcatgaagttattgagggtaaatacgcattgccaggtttaccagctacttttggtgacgcacaagatgctactacattggttgttcatttgtacgataactactcttcagttgcagctgatttgtcttactcaatcttcccaaagtacgatgctatcgttagatcagttaacgttacaaaccaaggtccaggcaacatcactatcgaagcattggcttctatctcaatcgatttcccatacgaagatttggatatggtttcattaagaggtgactgggcaagagaagctaacgttcaaagatcaaaggttcaatatggtgttcaaggttttggttcttcaactggttactcttcacatttgcataacccatttttagcaatcgttgatccagctactacagaatctcaaggtgaagcttggggttttaatttggtttacacaggttctttttcagcacaagttgaaaaaggttcacaaggttttactagagctttgttaggttttaatccagatcaattgtcttggaatttgggtccaggtgaaactttaacatcaccagaatgtgttgctgtttattctgataaaggtttgggttctgtttcaagaaagttccatagattgtacagaaaccatttgatgaagtctaagttcgctacatcagatagaccagttttgttaaattcatgggaaggtgtttacttcgattacaaccaatcttcaatcgaaactttagcagaagaatctgcagctttgggtgttcatttgttcgttatggatgatggttggtttggtgacaaatatccaagagtttctgataatgctggtttgggtgactggatgccaaatccagctagatttccagatggtttgacaccagttgttcaagatatcacaaatttgactgttaacggtacagaatcaactaagttgagattcggtatttgggttgaaccagaaatggttaacccaaattctacattgtaccatgaacatccagaatgggcattacatgctggtccatacccaagaactgaaagaagaaaccaattggttttgaatttggcattaccagctgttcaagatttcatcatcgatttcatgacaaatttgttgcaagatactggtatctcttacgttaagtgggataacaacagaggtattcatgaaacaccatctccatcaactgatcatcaatacatgttgggtttatacagagttttcgatactttgactacaagattcccagatgttttatgggaaggttgtgcttctggtggtggtagatttgatgctggcatgttacaatatgttccacaaatttggacatctgataacactgatgcaatcgatagaatcacaattcaatttggtacttcattagcttacccaccatctgcaatgggtgctcatttgtcagcagttccaaatgctcaaacaggtagaactgttccttttacttttagagctcatgttgctatgatgggtggttcttttggtttggaattagatccagctactgttgaaggtgacgaaattgttccagaattgttagcattggctgaaaaggttaacccaatcatcttgaacggtgacttgtacagattgagattaccacaagattcacaatggccagcagctttatttgtttctcaagatggtgcacaagctgttttgttttacttccaagttcaaccaaacgttaaccatgcagttccatgggttagattgcaaggtttagatccaaaagctgattatacagttgatggtgaccaaacatactctggtgcaactttaatgaatttgggtttacaatactcattcgatactgaatacggttctaaagttgttttcttggaaagacaataattaattaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatttcttccaaaaaaaaaaccgatgaattagtggaaccaaggaaaaaaaaagaggtatccttgattaaggaacactgtttaaacagtgtggtttccaaaaccctgaaactgcattagtgtaatacaagactagacacctcgatacaaataatggttactcaattcaaaactgcctttgaaacatcatgaaaactgtttcaccctctgtgaagcataaacactagaaagccaatgaagagctctacaagcctcatatgggttcaatgggtctgcaatgaccgcatacgggcttggacaattaccttctattgaatttctgagaagagatacatctgaccagcaatgtaagcagacaatcccaattctgtaaacaacctctttgtccataattccccatcagaagagtgaaaaatgccctcaaaatgcatgcgccactcccacctctcaactgcactgcgccacctctgagggtcctttcaggggtcgactaccccggacacctcgcagaggagcgaggtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttgcgttttcttttcaacttttttttactttttccttcaacagacaaatctaacttatatagatctatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagagatctgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattgatatcgtcaaggctgtccaagaggcaaccaatggtggtccacatggtgtcatcaatgtctctgtctctgaagctgcaatttctcaatcttgtgaatacgttagacctctaggtaaggttgttcttgttggtttaccagcaggcgcacaagtcaaaactggtgtctttgaagccgttgtcaagtctattgaaattaagggttcttatgtcggtaacagaaaggataccgccgaagcacttgacttctacactagaggcttggtcaagtctccattcaagattgtcggtttatccgaattgccaaaagtctttgaactcatggaacagggtaagattttaggtagaatggtcttagacacctccaaataagtctagagagtgtatacctccccgcttttgctgctactaattaatacccactattaatttccttctattacaaaacgcctctcagactcccacacacacacttacactagtggcctatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaac